(19) United States
(12) Patent Application Publication
(10) Pub. No.: US 2025/0316471 A1
(43) Pub. Date: Oct. 9, 2025

(54) ULTRAVIOLET LIGHT EMITTING ELEMENT AND ULTRAVIOLET LIGHT IRRADIATION DEVICE PROVIDED WITH THE SAME

(71) Applicant: SHIKOH TECH CO., LTD., Awaji-shi, Hyogo (JP)

(72) Inventors: Tsutae SHINODA, Akashi-shi (JP); Hitoshi HIRAKAWA, Takasago-shi (JP); Kenji AWAMOTO, Miki-shi (JP); Takefumi HIDAKA, Kobe-shi (JP); Tetsuya MAKINO, Kakogawa-shi (JP); Masayuki WAKITANI, Akashi-shi (JP)

(21) Appl. No.: 18/874,360

(22) PCT Filed: Jan. 25, 2024
(86) PCT No.: PCT/JP2024/002266

§ 371 Date: Dec. 12, 2024

(30) Foreign Application Priority Data
Feb. 6, 2023 (JP) 2023-016178

(51) Int. Cl. H01J 61/52; A61L 2/10; A61L 9/20; C09K 11/08; H01J 61/16; H01J 61/44

(52) U.S. Cl. CPC H01J 61/523; A61L 2/10; A61L 9/20; C09K 11/0872; H01J 61/16; H01J 61/44; A61L 2202/11; A61L 2209/212

(57) ABSTRACT

The present invention provides a safe ultraviolet light irradiation device that uses ultraviolet light safe to the human body and has a wide range of microorganism elimination and sterilization effects, and also provides an ultraviolet light emitting element provided with: an electrode substrate having a pair of electrodes; at least one cylindrical or flattened cylindrical glass tube disposed on the electrode substrate so as to face both of the electrodes, the glass tube having both end parts sealed; a xenon gas-containing discharge gas enclosed inside the glass tube and generating an electric discharge due to a voltage applied to the electrodes; and a phosphor layer formed on an inner surface of the glass tube and emitting light when excited by the electric discharge, wherein the glass tube is made of borosilicate glass or quartz glass; and the phosphor layer is made of a phosphate-based fluorescent material and has an emission spectrum having a peak half value width within a wavelength range of 50 nm or less with a peak at a wavelength of 203±10 inm on an illuminance basis.

### TECHNICAL FIELD OF THE INVENTION

The present invention relates to an ultraviolet light emitting element and an ultraviolet light irradiation device provided with the same. The present invention especially relates to an ultraviolet light emitting element that can realize disinfection devices by using ultraviolet light that is safe to the human body without using an expensive and high performance filter, for elimination of viruses/bacteria (including disinfection and sterilization) and deodorization and also relates to an ultraviolet light irradiation device provided with the above-described ultraviolet light emitting element.

In this specification, among ultraviolet rays (UV), deep ultraviolet rays (DUV) mean radiation in an ultraviolet region in a range of wavelengths from 200 nm to 300 nm; and vacuum ultraviolet rays (VUV) mean ultraviolet rays having wavelengths of 200 nm or less generated by noble gas discharge. LAFi (Luminous Array Film) refers to a surface light source structure in which a gas-discharging tube (glass tubule) is filled with phosphor and a discharge gas made of a mixture of neon and xenon, both ends of the glass tube are sealed, and multiple glass tubules are arranged on an electrode substrate, so as to emit light.

In this specification, the wavelength of ultraviolet light varies by about 10 nm, particularly on the short wavelength side, between cases where a “photon quantity (number of photons)” is used as a standard (here referred to as a photon wavelength (unit: pnm)) and cases where a “illuminance” is used as a standard (here referred to as an illuminance wavelength (unit: inm)). In other words, the characteristic based on the “photon quantity (number of photons)” is shifted to the long wavelength side by about 10 nm compared to the characteristic based on the “illuminance.” The reason for this is a relationship: illuminance (mW/cm²)=n×h×C/wavelength. n is the number of photons per unit, h is a Planck multiplier, and C is a speed of light. In other words, there is a relationship: illuminance ∝ number of photons/wavelength. Therefore, even with the same number of photons, the illuminance is higher for shorter wavelengths than for longer wavelengths, making an illuminance spectrum shift to the shorter wavelength side compared to the photon number spectrum. It is difficult to obtain a general-purpose measuring instrument with an illuminance standard of 200 inm or less, and measurements at 200 inm or less are generally made based on a photon quantity standard. The reason why the wavelength shifts is that transmissivity of glass tubules and sensitivity of a light receiving sensor of an illuminometer are not uniform with respect to the wavelength. Since the transmissivity of the glass tubules and the sensitivity of the light receiving sensor could decrease as wavelengths become shorter, the emission spectrum of a broadband light source shifts to longer peak wavelengths.

### BACKGROUND OF THE INVENTION

The microorganism elimination effects and the deodorization effects of ultraviolet light have been traditionally well known, and various devices for elimination of viruses/bacteria or disinfection using ultraviolet irradiation have been known (see, for example, PTL 1 to PTL 4). The below-listed PTL 1 suggests that ultraviolet rays with wavelengths of 200 to 230 inm are suitable for virus inactivation and optimal as not affecting human cells. In a device disclosed in PTL 1, an excimer lamp having a narrow peak wavelength of 222 inm is mentioned as a typical example of a light source that emits deep ultraviolet light in the above wavelength range.

### CROSS REFERENCES

### Patent Literature Documents

PTL 1: National Publication of Japanese Translation of PCT Application No. 2014-508612
PTL 2: PCT International Publication No. WO 2016/125708
PTL 3: Japanese Unexamined Patent Application Publication No. 2015-193521
PTL 4: PCT International Publication No. WO 2018/235723
PTL 5: PCT International Publication No. WO 2022/113943

### SUMMARY OF THE INVENTION

### Object of the Invention

However, the excimer lamp that emits deep ultraviolet rays with the wavelength of 222 inm uses krypton chloride (KrCl) gas, which is highly toxic, as a discharge gas; and when this lamp is broken, environmental problems would inevitably occur. Also, manufacturing sites of excimer lamps are required to have a special facility where is gas-proof. The KrCl excimer has a narrow emission wavelength width and thus has a problem that the excimer may not have an efficient disinfection effect against all kinds of bacteria and viruses. Namely, even though more efficient inactivation abilities against bacteria and viruses may be expected in the shorter wavelength region than 222 inm, the emission wavelength of this excimer lamp is limited almost to 222 inm. The excimer lamp thus is not expected to have inactivation abilities in the wavelength region shorter than 222 inm.

In a conventional surface-emitting ultraviolet light source device for disinfection disclosed in PTL2, a light source device with peak wavelengths exclusively in the vicinity of 260 inm was the limit of commercialization due to limitations in a glass material (traditional material) for gas-discharging tubes and limitations in a phosphor material. If the above-described light source device is used, deep ultraviolet light with wavelengths shorter than about 240 inm generated from the light source device is absorbed by the glass material used for tube envelope. Therefore, it has been desired to develop a new phosphor that generates ultraviolet light with shorter wavelengths, which is expected to have a high disinfection effect against viruses and bacteria, and also develop a new glass material that sufficiently allows deep ultraviolet light to pass therethrough. It was also necessary to develop new technology for preparing the new phosphor inside a glass tube for emitting deep ultraviolet light with shorter wavelengths with appropriate luminescence efficiency (light emission efficiency).

The inventors of the present invention have solved the above problems and provided a deep ultraviolet light irradiation device disclosed in PTL 5 as a disinfection device that has broad disinfection and elimination effects on many viruses and bacteria and yet is safe and has minimal impact on the human body. This deep ultraviolet light irradiation device is provided with a broadband light source (hereinafter referred to as 228B) that emits ultraviolet light with a peak wavelength of around 228 inm.

The deep ultraviolet light irradiation device of PLT 5 is different from the conventional method of emitting a wavelength of around 222 inm, which is considered to be useful for inactivating bacteria (germs), from an excimer lamp, and is based on a gas-discharging tube array-type surface-emitting light source device as a deep ultraviolet light source that utilizes a deep ultraviolet phosphor. The deep ultraviolet light irradiation device of PLT 5 also utilizes a deep ultraviolet phosphor layer that is excited by the discharge of xenon (chemical symbol: Xe) gas and has a wide emission spectrum in a wavelength range of at least 210 to 250 inm with a peak wavelength of around 228 inm. The deep ultraviolet light irradiation device of PL 5 further has an optical filter disposed opposite the light emitting surface (which is an arrangement surface formed of the light emitting surfaces of the arranged gas-discharging tubes) as necessary in order to reduce ultraviolet rays in a wavelength region of 240 inm or more in the emission spectrum of the phosphor layer, i.e., in order to suppress the transmission of the ultraviolet rays. This optical filter has a configuration of a dielectric multilayer filter formed on a surface of an ultraviolet light transmitting substrate made of quartz, and is arranged so that the ultraviolet light transmitting substrate faces the light-emitting surface of the surface-emitting ultraviolet light source device. The ultraviolet light transmitting substrate functions to adjust an incident angle of light emitted from the light-emitting surface to the dielectric multilayer filter.

The conventional deep ultraviolet light irradiation device disclosed in PTL 5 uses the optical filter to reduce effective emission illuminance (effective radiation illuminance), which is an index of safety, thereby providing a safer device. However, optical filters are expensive. Furthermore, with diffusive broadband waveforms such as 228B, it is not easy to effectively eliminate ultraviolet light in a wavelength region of 240 inm or more, which is less safe, because the optical filter is viewing angle dependent. Therefore, the structure of the optical filter and of the irradiation device had to be cogitated, making the optical filter more expensive and the structure of the irradiation device more complicated.

The present invention is to provide an ultraviolet light irradiation device that has wide disinfection and elimination effects against many viruses and bacteria and also reduces ultraviolet light in a wavelength region that has adverse effects on the human body-namely, the irradiation device that is safe and has minimal impact on the human body without basically using an optical filter that suppresses the transmission of ultraviolet light.

### BEST MODE FOR CARRYING OUT THE INVENTION

The present invention provides an ultraviolet light emitting element provided with:
an electrode substrate having a pair of electrodes;
at least one cylindrical or flattened cylindrical glass tube disposed on the electrode substrate so as to face both of the electrodes, the glass tube having both end parts sealed;
a xenon gas-containing discharge gas enclosed inside the glass tube and generating an electric discharge due to a voltage applied to the electrodes; and
a phosphor layer formed on an inner surface of the glass tube and emitting light when excited by the electric discharge,
wherein the glass tube is made of borosilicate glass or quartz glass, and the phosphor layer is made of a phosphate-based fluorescent material and has an emission spectrum having a peak half value width within a wavelength range of 50 nm or less with a peak at a wavelength of 203±10 inm on an illuminance basis.

An ultraviolet light irradiation device of the present invention is based on the use of a new broadband light source to be described below (which is hereinafter referred to as 203B) in order to effectively disinfect and minimize the impact on the human body. In the ultraviolet light irradiation device of the present invention, the peak wavelength is around 203±10 inm, preferably around 203 inm (200 inm to 208 inm) in a spectrum indicated by wavelength and irradiance. The ultraviolet light irradiation device of the present invention emits continuous ultraviolet light that has a wavelength range with a peak half value width of 50 nm, preferably a wavelength range with a peak half value width of 40 nm. The light source 203B is a gas-discharging tube array-type surface-emitting light source device that uses a phosphor in a similar way to 228B suggested by PLT 5. Compared to 228B, however, 203B is a light source that significantly reduces ultraviolet light in a wavelength region of 240 inm or higher, which is harmful to the human body. The phosphor of 203B (that is, a broadband wavelength centered at 203 inm (about 210 pnm)) is excited by vacuum ultraviolet light generated from Xe and emits the light. This light emission is close to broadband light centered around 180 pnm emitted from Xe through a glass tube. In order to effectively extract the 203B light, 180 pnm light must be extracted as well.

When a phosphor is irradiated with 147 pnm and/or 172 pnm vacuum ultraviolet light generated by an electric discharge in a glass tube and emitted, the ultraviolet light specific to the phosphor inside the glass tube will be referred to as ultraviolet light 1. The ultraviolet light 1 extracted to the outside through a wall of the gas-discharging tube (glass tubule) will be referred to as ultraviolet light 2. The ultraviolet light 1 and the ultraviolet light 2 have different spectral characteristics because of a wavelength region where the ultraviolet light 1 and the ultraviolet light 2 are absorbed when transmitted through glass, depending on the transmission characteristics of the glass. That is, depending on a material and a thickness of the glass tubule, the ultraviolet light 2 exhibits spectral characteristics, which are completely different from those of the ultraviolet light 1; and a spectrum can be understood only after the device is prepared. The ultraviolet light 203B of the present invention, which will be described below, refers to the aforementioned ultraviolet light 2.

As will be described in detail later, the inventors of the present invention have confirmed for the first time through extensive research and experimentation that 203B of the present invention exhibits capabilities of disinfection and elimination of viruses/bacteria equal to or greater than those of the conventional 228B. Also, the inventors of the present invention have evaluated effective emission illuminance (effective irradiance) used in safety standards and found that in the UV wavelength region of 240 inm and above, 203B that does not use an optical filter is as safe as or safer than the conventional 228B that uses an optical filter to reduce UV light, that is, to suppress UV light transmission. From these findings, the inventors of the present invention have found that the above-mentioned problems could be solved, leading to the present invention.

As another aspect of the present invention, the ultraviolet light irradiation device of the present invention does not basically require an optical filter; however, if higher safety is required, it is desirable to install an optical filter or to install a timer so as to control a driving time of the surface-emitting ultraviolet light source device. The optical filter installed in the present invention may be used that is similar to the one proposed in PLT 5. That is, this optical filter may be made from a dielectric multilayer filter formed on a surface of an ultraviolet light transmitting substrate made of quartz or the like and may be positioned so that the ultraviolet light transmitting substrate faces the light emitting surface of the surface-emitting ultraviolet light source device. The ultraviolet light transmitting substrate functions to adjust an incident angle of light emitted from the light emitting surface to the dielectric multilayer filter.

As another aspect of the present invention, glass with high transmission performance in a deep UV light region, such as borosilicate glass or quartz glass, is used as a glass material for an envelope to be used as the gas-discharging tube. Borosilicate glass has a property of absorbing ultraviolet rays in a wavelength range of 175 to 200 pnm and therefore can suppress the emission of ultraviolet rays of wavelengths of 190 pnm or less in a vacuum ultraviolet region, which generate ozone deleterious to the human body. This allows the borosilicate glass to emit vacuum ultraviolet rays (VUV: second peak) centered at 180 pnm, which are generated from the xenon (Xe) gas enveloped (sealed) therein as a discharge gas and extracted to the outside of the glass tubule. Quartz glass transmits vacuum ultraviolet rays better than borosilicate glass and can therefore emit vacuum ultraviolet rays (VUV: second peak) centered around 173 pnm. The present invention can utilize a surface-emitting ultraviolet light source device formed of a gas-discharging tube array that can efficiently emit deep ultraviolet light included in 203B particularly deep ultraviolet light in a wavelength region of 200 inm to 250 inm that is expected to be most effective for disinfection and elimination of viruses/bacteria—from a phosphor layer.

Another aspect of the present invention is that by using 203B as a broadband light source that emits ultraviolet rays, ultraviolet rays in a wavelength region of 240 inm or more that have adverse effects on the human body are significantly reduced. Furthermore, the present invention provides an ultraviolet light irradiation device in which a thickness of the glass tube (glass thickness) forming the envelope of the gas-discharging tube is adjusted so as to limit the radiation of ultraviolet rays generated thereinside on the short-wavelength side. In other words, 203B emits ultraviolet rays in the deep ultraviolet region with a wide wavelength range of 200 inm to 250 inm, which is expected to be most effective for disinfection and elimination of viruses/bacteria, but also greatly reduces the emission of ultraviolet rays at wavelengths of 240 inm or more, which are harmful to the human body. Furthermore, ultraviolet rays in a vacuum ultraviolet region with wavelengths of 190 pnm or less, which generate ozone that is harmful to the human body, can be suppressed by adjusting a thickness of the glass tube according to its glass material. For example, in a case of glass tubes made of borosilicate glass, the glass thickness should be in a range of around 60 to 300 μm to effectively suppress the emission of ultraviolet rays in a vacuum ultraviolet region with wavelengths of 200 inm or less—especially ultraviolet rays in a vacuum ultraviolet region with wavelengths of 190 pnm or less that generate ozone harmful to the human body—from the glass tube.

As another aspect of the present invention, the inventors of the present invention have conducted extensive research and experiments and found that the 203B broadband light source used in the ultraviolet light irradiation device of the present invention has many advantages as described above but also has a problem of being “highly temperature dependent.” For this reason, the ultraviolet light irradiation device of the present invention is desirably characterized by having a heat-releasing mechanism or a cooling device that suppresses or controls a temperature rise of the light source device. The heat-releasing mechanism or the cooling device may be any of those that utilize a specially designed substrate or device structure or those that are provided with a device or an element that actively promotes cooling. Specifically, for example, a slit substrate in which a through-hole part such as a slit is formed may be used as an electrode substrate constituting the light source device. A heat sink, which is made of ceramic, aluminum, or the like, and/or a Peltier element (Peltier device) or a vapor chamber may be attached to the back of the electrode substrate. Alternatively, a cooling fan may be installed on the back of the light source device. The heat releasing mechanism or the cooling device is not limited to these; however, a combination of these specific structures can be used so as to achieve better effects. When using highly conductive heat sinks made of aluminum or the like, it is necessary to attach the heat sinks on the back surface side of the electrode substrate and also at separate positions corresponding to the electrodes, respectively, in order to prevent electrical shorts.

Another aspect of the present invention is characterized in that quartz glass or borosilicate glass is disposed at a position 1 mm or more away from the surface of the light source device, and the atmosphere is sealed in a glass tube at its periphery. Ozone (molecular formula: O₃) may be effectively generated to make active use of ozone; however, its generation must be suppressed if ozone is not to be used, given that high concentrations of ozone are harmful (hazardous). By adopting such a structure, ozone can be enclosed in a sealed space; and diffusion of ozone to the outside can be suppressed (or prevented). In addition, since vacuum ultraviolet rays are absorbed by oxygen to generate ozone in a void between the surface of the light source device and the quartz glass or the borosilicate glass, short wavelength ultraviolet light will be cut off in this void.

As a further variation of this, it is also possible to cut off short wavelength ultraviolet light by using a sealed panel prepared by placing two sheets of quartz glass or glass that transmits ultraviolet light in close contact with the surface of the light source device with a void of about 1 mm to 5 mm between the two glass sheets and by sealing the periphery of the light source device and the two glass sheets.

### Advantageous Effects of the Invention

The present invention makes it possible to realize a deep ultraviolet light irradiation device with wide elimination/disinfection effects on viruses/bacteria (microorganisms) using ultraviolet light that is safe for the human body, without the use of an expensive and high-performance optical filter. Even when the ultraviolet light irradiation device is used by people who are unfamiliar with handling ultraviolet light or even when the ultraviolet light irradiation device is used in an environment where is not designed and controlled to prevent ultraviolet light in a wavelength region harmful to the human body from leaking out, it becomes possible to handle the ultraviolet light irradiation device with high safety. The ultraviolet light irradiation device of the present invention broadly covers a wavelength range of 200 inm to 250 inm, which is expected to be most effective for disinfection and elimination of viruses/bacteria, but radiates significantly less ultraviolet light in a deep ultraviolet region with wavelengths of 240 inm or more, which are harmful to the human body.

### BRIEF DESCRIPTION OF THE DRAWINGS

FIG. 1A shows a diagrammatic view of a configuration of a gas-discharging tube array-type surface-emitting ultraviolet light source device in accordance with First Embodiment in an ultraviolet light irradiation device of the present invention.

FIG. 1B shows a diagrammatic view of a configuration of the gas-discharging tube array-type surface-emitting ultraviolet light source device in accordance with First Embodiment in the ultraviolet light irradiation device of the present invention.

FIG. 1C shows a diagrammatic view of a configuration of the gas-discharging tube array-type surface-emitting ultraviolet light source device in accordance with First Embodiment in the ultraviolet light irradiation device of the present invention.

FIG. 1D shows a perspective view of a heatsink as a heat-releasing mechanism.

FIG. 1E shows a perspective view of another heatsink as a heat-releasing mechanism.

FIG. 1F shows a perspective view of a Peltier element as a cooling device.

FIG. 1G shows a perspective view of a complex comprising the heat-releasing mechanism and the cooling device.

FIG. 2 is a graph showing light transmission characteristics of borosilicate glass based on a photon standard.

FIG. 3 is a graph showing emission spectra of ultraviolet light source devices.

FIG. 4 is a graph showing action functions with respect to wavelengths.

FIG. 5 is a graph showing deactivation rates of the ultraviolet light source devices.

FIG. 6 is a graph showing the variation in illuminance depending on lighting time of each ultraviolet light source.

FIG. 7 is a graph showing the variation in illuminance depending on lighting time of each ultraviolet light source.

FIG. 8 is a graph showing a change in illuminance with respect to a change in temperature in a 203B light source.

FIG. 9 shows a cross-section view of a structure of an ultraviolet light irradiation device in accordance with Second Embodiment.

FIG. 10 is a comparison Table showing effectual irradiance illuminance in different categories.

FIG. 11A shows a diagrammatic view of an assembled structure of an ultraviolet light irradiation device in accordance with Third Embodiment.

FIG. 11B shows a diagrammatic view of an assembled structure of the ultraviolet light irradiation device in accordance with Third Embodiment.

FIG. 12 shows a diagrammatic view of an assembled structure of an ultraviolet light irradiation device of the present invention in accordance with Fourth Embodiment.

FIG. 13 is a graph showing emission spectrum characteristics of the 203B light source of the present invention.

FIG. 14 shows a diagrammatic view of an assembled structure of an ultraviolet light irradiation device in accordance with Fifth Embodiment.

FIG. 15A is a Table showing an increase in surface temperature of the 203B light source under various conditions.

FIG. 15B is a graph based on the Table shown in FIG. 15A.

### DETAILED DESCRIPTION OF THE INVENTION

In the following, this invention will be described in detail through the use of the drawings. The following descriptions should be recognized as exemplifications in all respects, and should not be interpreted to limit this invention.

### First Embodiment

Basic Structure and Drive Principle of Surface-Emitting Ultraviolet Light Source Device A gas-discharging tube array of the present invention used as a surface-emitting ultraviolet light source device is basically the same in structure as a tube array disclosed in the above-listed PTL 5, except for a material for a phosphor and glass tubules used for the tube array. The present invention utilizes, as a light source of deep ultraviolet light within a wavelength band (185 pnm to 240 inm) effective for virus inactivation, the gas-discharging tube array-type surface-emitting ultraviolet light source device characterized by not using highly toxic KrCl gas or environmentally problematic mercury.

### Gas-Discharging Tube

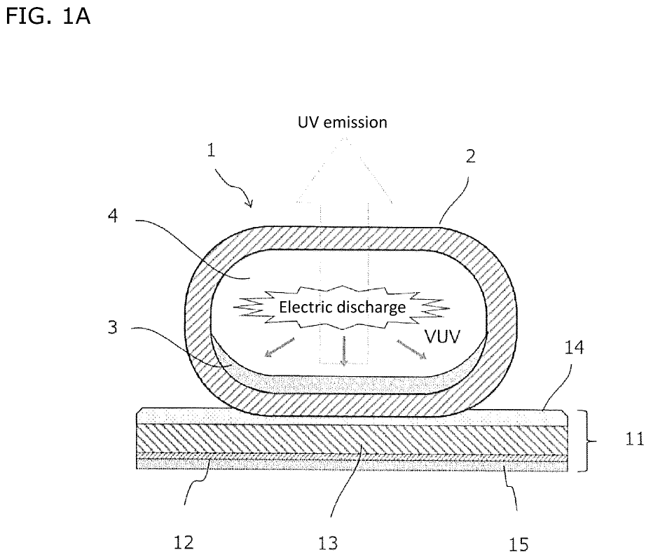

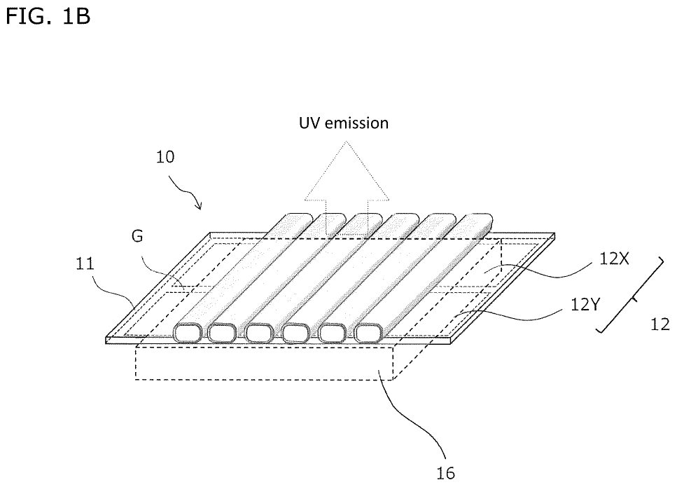

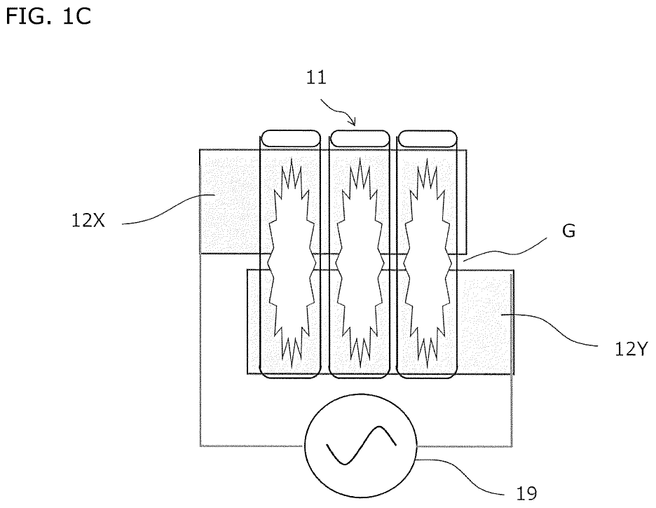

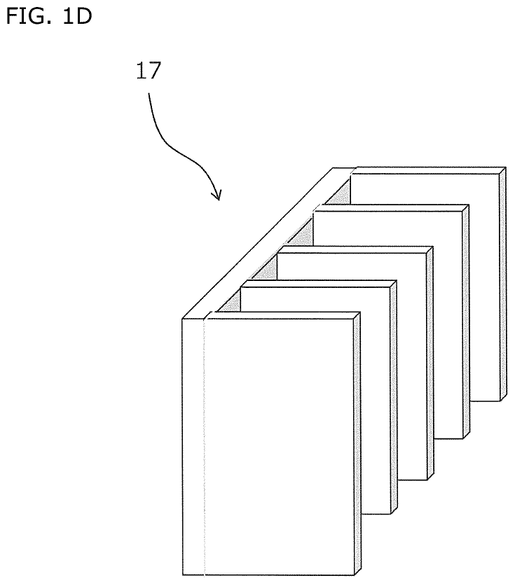

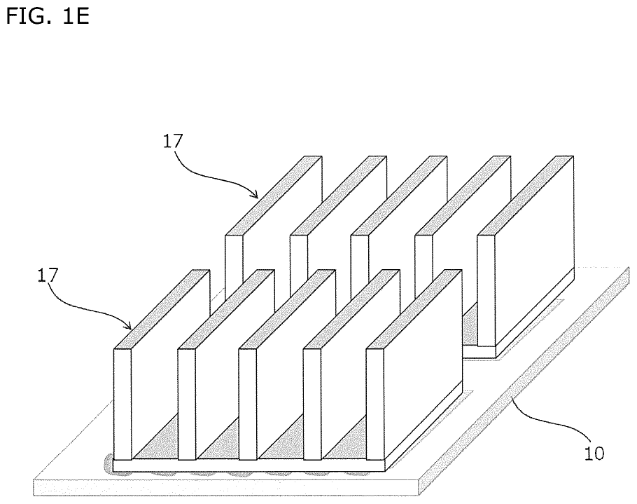

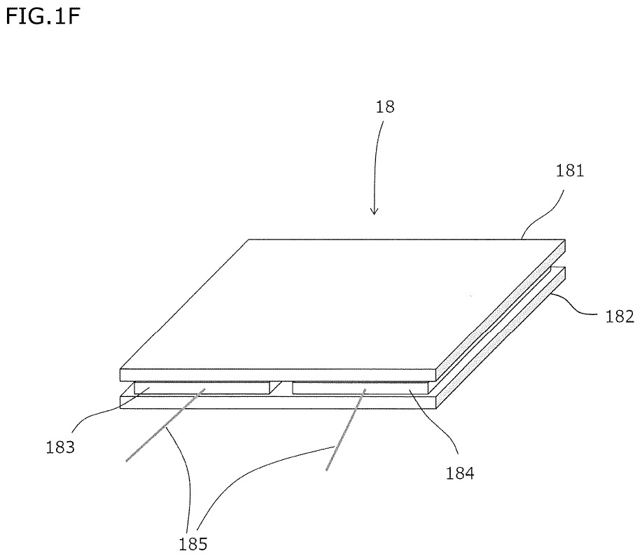

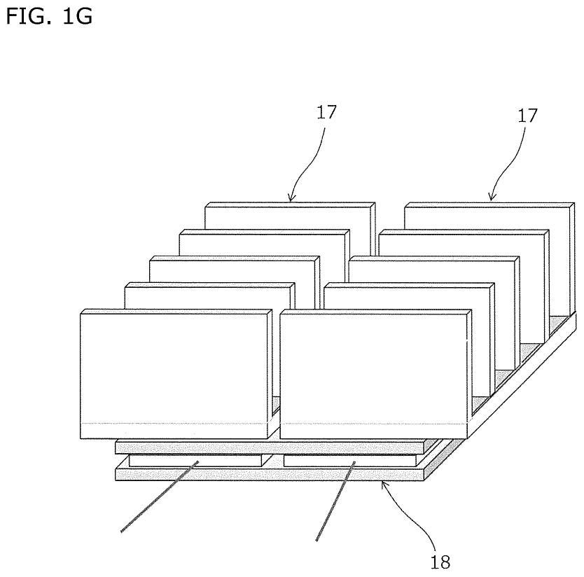

FIG. 1A to FIG. 1C show diagrammatic views of configurations of a gas-discharging tube array-type surface-emitting ultraviolet light source device in accordance with First Embodiment in an ultraviolet light irradiation device of the present invention; and FIG. 1A shows a cross-section view of a gas-discharging tube, which functions as a deep ultraviolet light emitting element. A gas-discharging tube 1 is mainly formed of an 8 cm long glass tubule 2 having a flat-oval cross-section with a major axis of about 2 mm and a minor axis of about 1 mm as an example, and has a deep ultraviolet phosphor layer 3 on an inner surface of the tubule on the rear surface side that is on the opposite side where emits ultraviolet light. Also, the gas-discharging tube 1 is configured to enclose a discharge gas 4 inside the glass tubule 2, the discharge gas containing noble gases that emit vacuum ultraviolet light, such as a mixed gas of neon (chemical symbol: Ne) and xenon (Xe), a mixed gas of helium (chemical symbol: He) and xenon (Xe), a mixed gas of argon (chemical symbol: Ar) and xenon (Xe), or a mixed gas of krypton and xenon, and is configured to seal both sides of the gas-discharging tube.

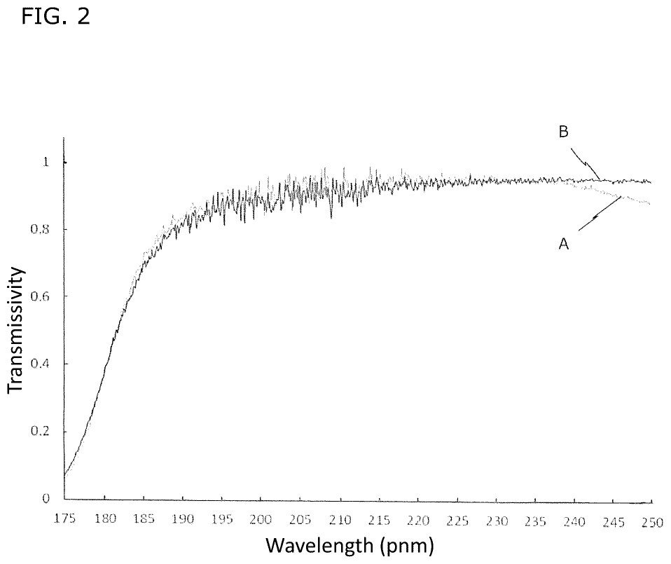

As a material for the glass tubule 2, inexpensive borosilicate-based glass or highly UV-transmitting soft glass is used when generating DUV only. When generating VUV and DUV simultaneously, the following glasses are suitable: ultraviolet-transmissible borosilicate glass having a borosilicate-based structure to which a minute amount of fluorine or the like is added so as to improve ultraviolet transmissivity (transmittance); and quartz glass. As the ultraviolet-transmissible borosilicate glass, the following glass, for example, may be used: glass known by the trade name BU-41 (Nippon Electric Glass Co., Ltd.) or glass known by SCHOTT 8337B (SCHOTT). FIG. 2 indicates light transmission characteristics of borosilicate glass on a photon basis (transmissivity of ultraviolet light transmitting glass tube). In FIG. 2, the horizontal axis indicates wavelengths (pnm) and the vertical axis indicates transmissivity. Borosilicate glass has a characteristic of absorbing ultraviolet rays in a wavelength range of 175 to 200 pnm and thus is capable of suppressing emission of ultraviolet rays in a vacuum ultraviolet region of 190 pnm or less, which generates ozone that is deleterious to the human body, by controlling a thickness of the glass. In FIG. 2, lines A and B respectively indicate light transmissivities of ultraviolet light transmitting glass tubes made of the following glasses—trade names: BU-41-2 and BU-41-3 manufactured by Nippon Electric Glass Co., Ltd. Quartz glass, which is expensive, as a matter of course, but has excellent ultraviolet transmittance, may also be used. When preparing a glass tubule, a glass tube made of the above-described ultraviolet-transmissible borosilicate glass may be drawn (redrawn) to a thickness (glass thickness) of 200 μm or less, preferably to about 100 μm, so as to thin the glass thickness, thereby obtaining a glass tubule 2 that transmits light from a vacuum ultraviolet region around a wavelength of about 170 nm to a deep ultraviolet region around a wavelength of about 280 nm with a transmissivity of 80% or more. This glass tubule 2 is made of the borosilicate glass that transmits 80% or more of light at a wavelength of 200 pnm and transmits 45% or less of light at a wavelength of 180 pnm, on a photon quantity (amount) basis. However, a glass thickness of 50 μm or less is undesirable due to lack of strength and risk of breakage.

The deep ultraviolet phosphor layer 3 newly used for the present invention is a phosphate-based phosphor made of ScPO₄, which is a phosphate of scandium (chemical symbol: Sc) having an emission spectrum peak in the vicinity of a wavelength of 203 inm when excited by, for example, vacuum ultraviolet light.

As an excitation source of vacuum ultraviolet irradiation on the phosphor layer 3, any light source may be used as long as this light source is capable of emitting vacuum ultraviolet light having excitation wavelengths of 200 nm or less. For example, the following may be used as the excitation source: krypton (Kr) gas (147 nm wavelength), xenon (Xe) gas (173 nm wavelength), neon (Ne) (143 nm wavelength), or a mixed gas of these gases.

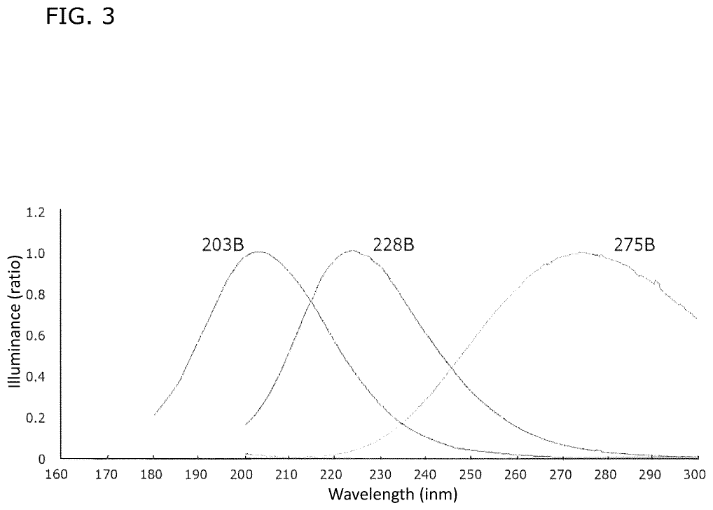

FIG. 3 is a graph showing emission spectra of UV light sources.

As indicated as 203B in FIG. 3 as an example, an emission spectrum of the surface-emitting ultraviolet light source used in the ultraviolet light irradiation device of the present invention has a peak at a wavelength of 203±10 inm, preferably around 203 inm (200 to 208 inm), due to the above-mentioned phosphor and glass tubule. The ultraviolet light irradiation device of the present invention emits ultraviolet light having a wavelength range with a peak half value width of 50 inm, preferably a wavelength spread of about 40 inm. Although 203B has a limit to measurement in a low wavelength region with a peak of around 203 inm, 203B has a wide continuous wavelength width from approximately 170 pnm to 260 inm and emits effective vacuum ultraviolet light and deep ultraviolet light at least in a range of 180 pnm to 235 inm.

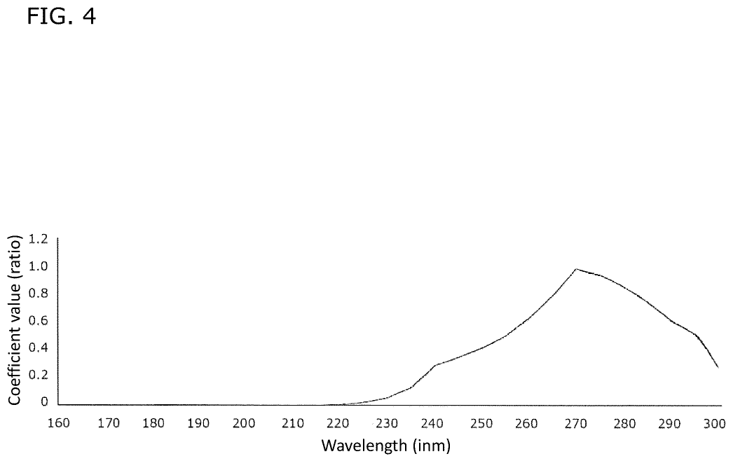

In addition to 203B, which is the light source of the present invention, FIG. 3 shows the emission spectra of the following light sources for comparison: 228B having a peak wavelength of around 228 inm; and a broadband light source emitting ultraviolet light with a peak wavelength of around 275 inm (hereinafter, this light source will be referred to as 275B). In FIG. 3, wavelengths are shown on the horizontal axis (unit: inm), and illuminance at each wavelength is shown on the vertical axis as a ratio to illuminance at a peak wavelength. 228B and 275B have basic structures similar to that of 203B. FIG. 4 shows an action function as coefficients that indicate adverse effects (degree of inhibition) of each wavelength (horizontal axis: unit in inm) of ultraviolet light (UV radiation) on the human body. The action function indicates “1” as a maximum coefficient value; and the lower the coefficient value, the less the adverse effects, which means safer. As shown in FIG. 4, the action function reaches its maximum at around 270 inm—that is, a coefficient value is “1”; and the long wavelength side reaches around 30% (coefficient value: 0.3) at around 300 inm, and the short wavelength side reaches around 30% at around 240 inm. Furthermore, the action function rapidly decreases on the short wavelength side after around 240 inm.

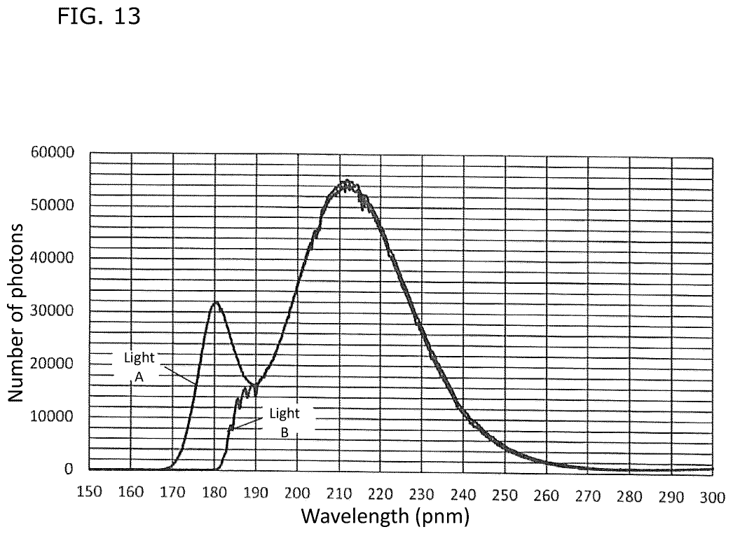

The emission spectra in FIG. 3 were measured under the following conditions. A measuring instrument used was a Maya 2000 Pro manufactured by Ocean Photonics. The wavelength region of the spectra of 200 inm or more was measured in illuminance mode. Since the wavelength region of 200 inm or less in 203B is the limit of measurement in the illuminance mode of this measuring instrument, the wavelength region was measured in photon mode (see FIG. 13) and calculated from a correlation of waveforms in the wavelength region of 200 inm or more between the illuminance mode and the photon mode. 203B was placed approximately 1 mm away from a measurement head of the measuring instrument; and the measurement head and 203B were placed in a container capable of sealing nitrogen. The illuminance mode was measured in an air atmosphere, and the photon mode was measured in a nitrogen atmosphere.

Returning to FIG. 3 and comparing the illuminance spectra, 203B is a broad (broadband) emission spectrum with a peak of around 203 to 204 inm. In 203B, an emission spectrum near 240 inm is 20% or less of the peak (about 1/10), and an emission spectrum near 250 inm is 10% or less of the peak (about 1/20); and the emission spectrum becomes smaller as the wavelength becomes longer. In contrast, in 228B, an emission spectrum near 240 inm is about 60% of a peak, and adverse effects on the human body (degree of obstruction) are significantly higher than in 203B. In 275B, illuminance of an emission spectrum is highest in a wavelength region in the vicinity of 270 inm where an action function is highest.

In this way, the inventors of the present invention have clarified for the first time that the ultraviolet light source 203B used in the deep ultraviolet light irradiation device of the present invention has significantly improved safety thereof, compared to the conventional ultraviolet light sources. Furthermore, the inventors of the present invention have compared 203B of the present invention with the conventional 228B in terms of sterilization performance, and have confirmed for the first time that 203B exhibits sterilization and disinfection capabilities similar to those of 228B.

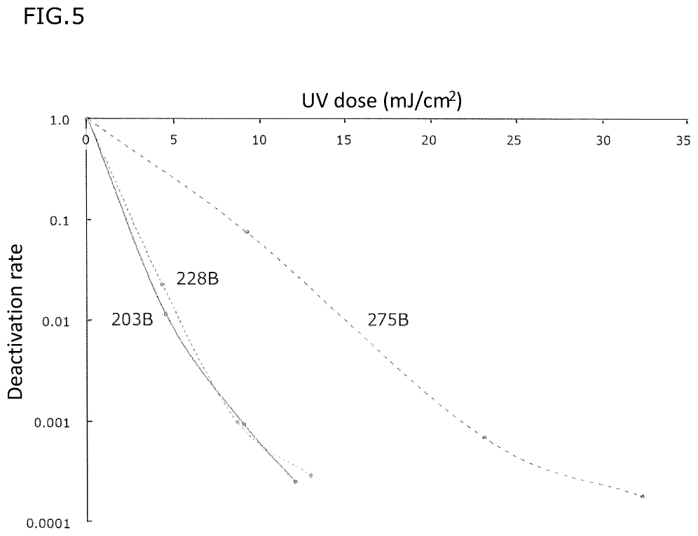

FIG. 5 shows a graph comparing the bactericidal ability of each light source as deactivation rates to demonstrate the above-mentioned results, and shows the results of Bacillus subtilis var. natto inactivated by irradiating this bacterium with ultraviolet light emitted from the three types of light sources—203B, 228B, and 275B—shown in FIG. 3. In FIG. 5, deactivation rates in the vertical axis indicate values obtained by dividing “the number of bacteria remaining” after UV irradiation by “the number of bacteria before UV irradiation” under each condition (the above-described three types of light sources). The horizontal axis in FIG. 5 indicates UV dose (mJ/cm²).

As is clear from FIG. 5, deactivation rates at the same UV dose are the lowest when the UV is emitted from 203B, and it is recognized that 203B has inactivation power equal to or greater than that of 228B and 275B. When the three types of light sources 203B, 228B, and 275B were compared to each other, the order shown in FIG. 5 became clear to the inventors of the present invention; in other words, the inventors have found for the first time through this experiment that 203B has inactivation power that is almost the same as 228B, but is greater than 275B. The UV doses for 99.9% inactivation in this experiment are about 8 mJ/cm² for both 203B and 228B and 21.5 mJ/cm² for 275B.

The sterilization experiments shown in FIG. 5 were carried out as follows. The bacterium used was Bacillus subtilis var. natto. Approximately 6 million Bacillus subtilis var. natto were dispersed in an aqueous solution, and the aqueous solution was applied to ten (10) culture beds (Sanispec stamps). One of the ten culture beds was left unexposed to UV light; and the other nine (9) culture beds were divided into three (3) groups, and the three groups were exposed to (or irradiated with) UV light emitted from 203B, 228B, and 275B light sources, respectively. 203B emitted 3 types of radiation: 4.5 mJ/cm², 9 mJ/cm², and 12 mJ/cm²; 228B emitted 3 types of radiation: 4.3 mJ/cm², 8.6 mJ/cm², and 12.9 mJ/cm²; and 275B emitted 3 types of radiation: 9.2 mJ/cm², 23 mJ/cm², and 32 mJ/cm². The ten culture beds, including the one culture bed that was not irradiated with ultraviolet light, were left in a thermostatic chamber at 30° C. for 20 hours to culture the bacterium. After that, photographs of each culture bed were taken; and the number of remaining bacteria was checked (counted). An inactivation rate for each condition was calculated by dividing the number of bacteria under each condition by the number of bacteria on the culture bed not exposed to the UV rays. Note that disinfection effects of UV rays tend to be similar for bacteria and viruses, and the effects can be represented by bacteria.

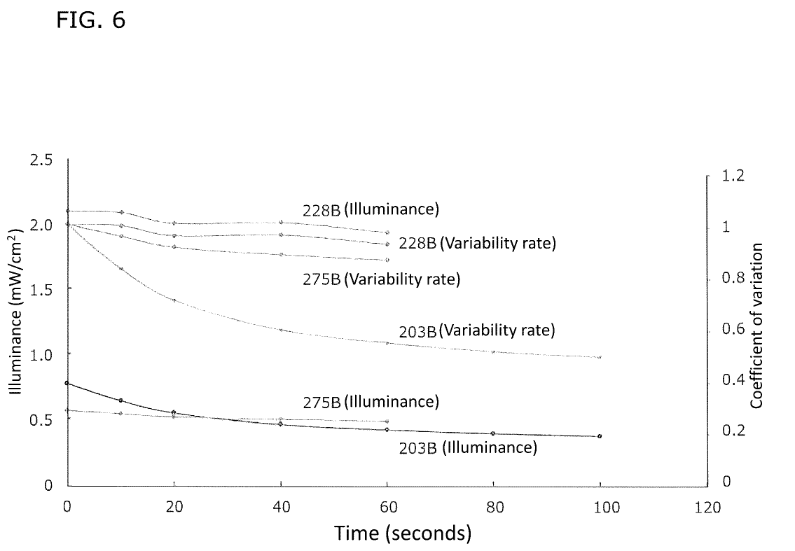

As described above, 203B, which is the light source used in the deep ultraviolet light irradiation device of the present invention, is the most efficient light source with higher safety than the previously suggested 228B and 275B and having the same as or better inactivation power than 228B and 275B. This fact was first revealed by the inventors of the present invention through their research and experiments. While 203B has many advantages as described above, it was ascertained that its illuminance varies greatly depending on lighting time. FIG. 6 is a graph showing the variation in illuminance depending on lighting time of each ultraviolet light source, and shows a relationship between the lighting time and the illuminance in each of the light sources 203B, 228B, and 275B. In FIG. 6, the horizontal axis indicates lighting time (seconds); the vertical axis on the left indicates illuminance (mW/cm²); and the vertical axis on the right indicates coefficient of variation (variability rate). The graph of FIG. 6 shows as graph lines, from top to bottom, illuminance of 228B, variability rates of 228B, variability rates of 275B, variability rates of 203B, illuminance of 275B, and illuminance of 203B. As is clear from the graph, compared to 228B and 275B, 203B has a rapid drop in illuminance over a short period of time and a larger rate of variability.

The measurements of measured values shown in FIG. 6 were made as follows: Each of 203B, 228B, and 275B was configured to have an irradiation surface of 8 cm×6 cm and was driven using an inverter made by the inventors; a voltage of 12 V was applied to the inverter; and the electric current was 1.4 A. Illuminance was measured with a simple handy-type illuminance meter at a distance of 5 mm from the light source surface in a non-contact manner. Since this simple handy-type illuminance meter cannot capture the entire wavelength of each light source, the illuminance is a relative value; however, this does not interfere with the measurement of the relative value of illuminance that fluctuates with lighting time.

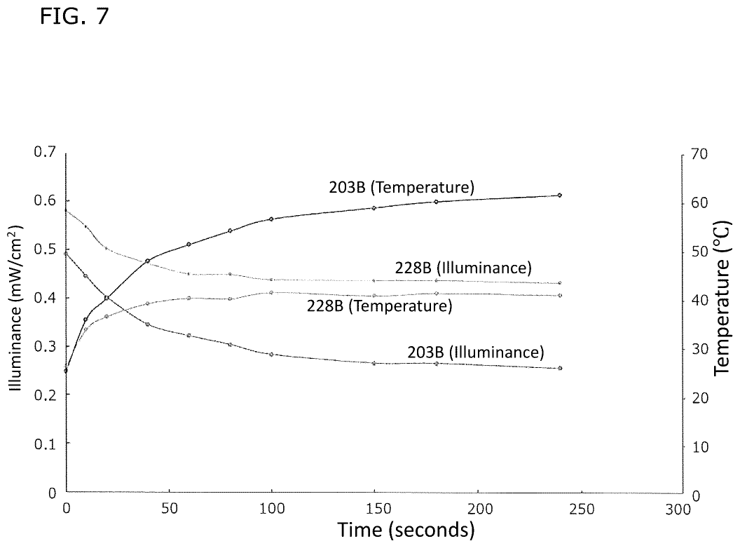

FIG. 7 is a graph showing time variation of illuminance and temperature with lighting time. In FIG. 7, the horizontal axis indicates lighting time (seconds); the vertical axis on the left indicates illuminance (mW/cm²); and the vertical axis on the right indicates temperature (° C.). The graph shows as graph lines, from top to bottom, changes in temperature of 203B, changes in illuminance of 228B, changes in temperature of 228B, and changes in illuminance of 203B; and all of the graph lines show the case where the light source devices 203B and 228B are cooled by blowing air on the light source devices. As is clear from FIG. 7, the temperature rises and the illuminance falls as the lighting time of both ultraviolet light sources 203B and 228B passes. It is thus recognized that the fall in illuminance depends on the temperature rise as the lighting time passes and that the changes (fluctuations) in temperature rise and illuminance fall are greater for 203B than for 228B as the lighting time passes. As shown in FIG. 7, when cooling is performed by blowing air, the rise in temperature is less and the fall in illuminance is less than in the case of FIG. 6 where no cooling is performed.

The measurements of measured values shown in FIG. 7 were made as follows: Each of 203B and 228B was configured to have an irradiation surface of 8 cm×6 cm, in the same way as in FIG. 6. Temperature and illuminance were measured at the same time: Illuminance was measured as described above; and temperature was measured in a non-contact manner using an infrared thermometer for a specified exposure time.

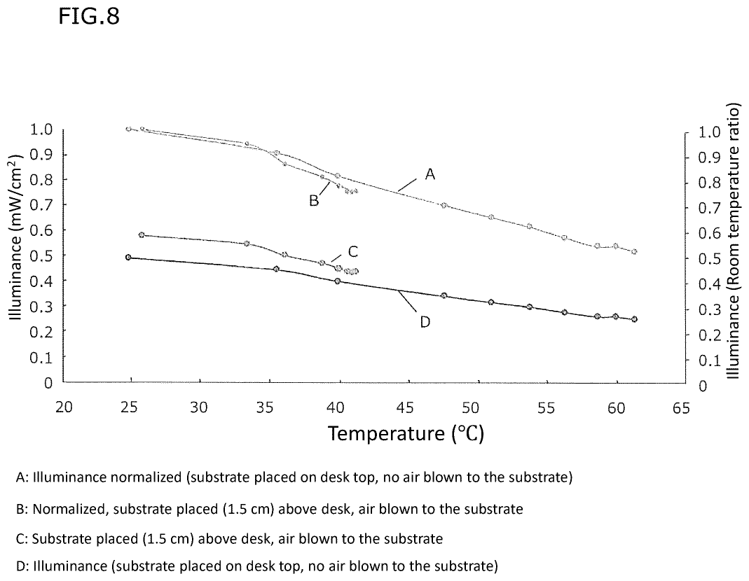

FIG. 8 is a graph showing a change in illuminance with respect to a change in temperature in the 203B light source. In FIG. 8, the horizontal axis indicates temperature (° C.); the vertical axis on the left indicates illuminance (mW/cm²); and the vertical axis on the right indicates room temperature ratio of illuminance. For the measurements in graphs D and C shown in FIG. 8, a 203B light source having a slit substrate with an 8×6 cm irradiation surface is used. FIG. 8 shows a comparison between the following two 203B light sources: The one is placed on a desk, driven, and allowed to rise in temperature without being cooled to measure temperature and illuminance (graph D); and the other one was placed 1.5 cm above the desk to facilitate cooling by wind (about 4 m/sec) to measure temperature and illuminance (graph C). The light source of graph D is not cooled, and its temperature rises to 62 degrees, whereas the light source of graph C is cooled by air blown by a cooling fan, and its temperature rises only to 42 degrees. In both cases, the illuminance decreases as the temperature rises. Graphs A and B normalize the relationship between temperature and illuminance in graphs D and C, respectively, to the illuminance at 25° C. The fact that graphs A and B show almost the same curves for temperature rise up to 42 degrees indicates that the illuminance is a function of the temperature. It can be seen from these curves that by keeping the temperature rise within 10 degrees, the illuminance can be kept within a 10% fluctuation.

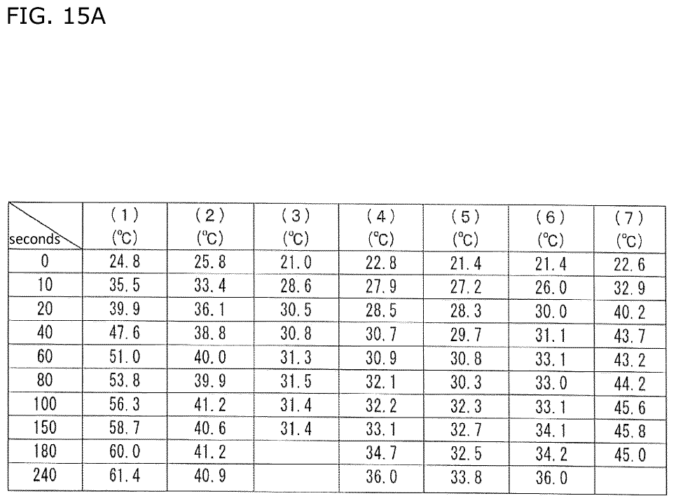

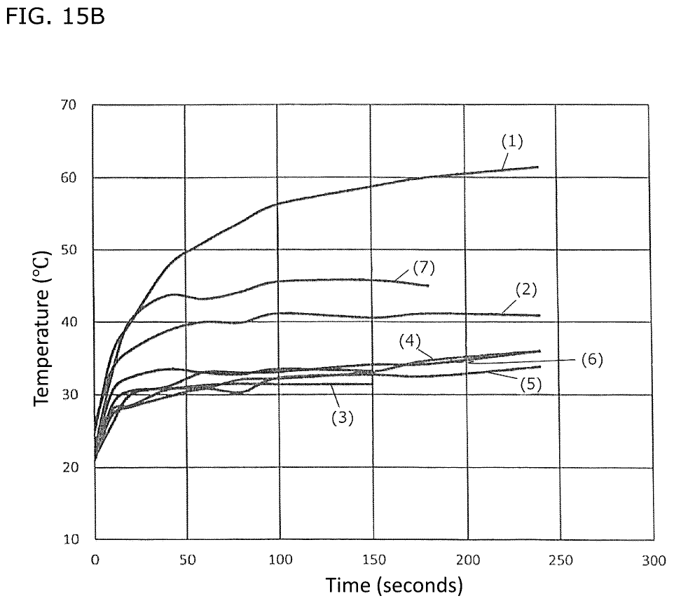

As is clear from FIG. 8, it is essential for the 203B light source to suppress temperature rise for stable operation. It is thus necessary to cool the 203B light source by taking measures such as adopting a slit substrate, air-cooling the light source with a cooling fan, adopting a heat sink, water-cooling the light source, and attaching a Peltier element, so as to suppress the temperature rise. The 203B light source may have the following structures, either alone or in combination, on its rear surface: a heat-releasing mechanism, such as a heat sink; and a cooling device, such as a Peltier element or a vapor chamber, to release heat from or cool the light source. By suppressing the temperature rise in this way to about 35 degrees or less, the fluctuation in illuminance can be suppressed to within 90% of the initial value; and the UV dose can be stabilized. This will be described in detail later with reference to FIGS. 15A and 15B and the like.

### Surface-Emitting Ultraviolet Light Source Device

With reference to FIG. 1A to FIG. 1C, the surface-emitting ultraviolet light source device will be described again. As shown in FIG. 1A and FIG. 1B, the gas-discharging array-type surface-emitting ultraviolet light source device 10 is formed such that a plurality of gas-discharging tubes 1 as ultraviolet light-emitting elements are arranged in parallel with each other on an electrode substrate 11 having a pair of electrodes 12 (a pair of an electrode 12X and an electrode 12Y). The electrode substrate 11, for example, comprises a polyimide-based insulating substrate 13 as a main body, supports an array of the gas-discharging tubes 1 by using an adhesive layer 14 placed on the insulating substrate, and comprises the electrode pair 12 placed on the opposite side of the adhesive layer and on a lower surface of the insulating substrate. The electrode pair 12 is also covered with an electrode-covering layer (insulating layer) 15. The electrode substrate 11 includes the electrode pair 12, the insulating substrate 13, the adhesive layer 14, and the electrode-covering layer 15.

In order to enhance the heat-releasing effect or the cooling effect of the surface-emitting ultraviolet light source device 10, a heat-releasing mechanism or a cooling device 16 is selectively provided, as necessary, on the lower surface side (which is on the rear surface side of the electrode-covering layer (insulating layer) 15 of the electrode substrate 11) of the pair of electrodes 12 that constitute the surface-emitting ultraviolet light source device 10. As the heat-releasing mechanism 16, a heat sink 17 is used as shown, for example, in FIG. 1D and FIG. 1E. The heat sink 17 is desirably made of ceramic or aluminum, but is not limited to this. When a metal material such as aluminum is used for the heat sink 17, it is necessary to install the heat sink 17 on the exposed rear side of the electrode-covering layer 15 (see FIG. 1A) and also to divide the heat sink into several pieces (at least two pieces as shown in FIG. 1E) so as to correspond to the electrode 12X and the electrode 12Y (see FIG. 1), respectively, in order to prevent electrical shorts.

To further enhance the heat-releasing effect or the cooling effect of the surface-emitting ultraviolet light source device 10, a Peltier element (Peltier device) 18 as shown in FIG. 1F may be selectively used as the cooling device 16 as necessary. In this case, the Peltier element 18 is installed in such a way that its heat absorbing plate 182 is in contact with the rear surface of the electrode-covering layer 15 constituting the electrode substrate 11. In the Peltier element 18, metal electrodes, which are connected with power supply lines (feed lines) 185, and semiconductors—an N-type semiconductor 183 and a P-type semiconductor 184—are alternately connected between a heat-releasing plate 181 and a heat-absorbing plate 182 placed above and below each other. When an electric current flows from the power supply lines 185, heat absorbed by the heat-absorbing plate 182 by a Peltier effect is transferred to the heat-releasing plate 181 and released from the heat-releasing plate 181. This allows the heat generated in the surface-emitting ultraviolet light source device 10 to be released effectively into the atmosphere via the heat-absorbing plate 182 and the heat-releasing plate 181. The cooling device 16 is not limited to the Peltier element; and instead of the Peltier element, a vapor chamber, for example, may be used. As shown in FIG. 1G, by attaching the heatsinks 17 to the heat-releasing plate 181 side of the Peltier element 18 and using the two combined as a complex, the heat on the heat-releasing plate 181 side of the Peltier element 18 can be efficiently released; and the function of the Peltier element 18 can be exerted at the maximum. This can further enhance the heat-releasing effect or the cooling effect of the surface-emitting ultraviolet light source device 10. Instead of the heatsink 17, the vapor chamber may be attached to the heat-releasing plate 181 side of the Peltier element 18 so as to effectively release the heat on the heat-releasing plate 181 side of the Peltier element 18. When the heat sink 17 and/or the Peltier element 18 is used, the surface-emitting ultraviolet light source device 10 will be hereinafter referred to as including the heat sink 17 and/or the Peltier element 18.

The gas-discharging array-type surface-emitting ultraviolet light source device 10 shown in FIG. 1B has a structure in which the pair of electrodes 12 is formed below the insulating substrate 13, and the adhesive layer 14 functioning as an insulating layer on the insulating substrate 13 supports the array of the gas-discharging tubes 1. The electrode pair 12 is formed of the electrode 12X and the electrode 12Y that are arranged on bottom surfaces of the gas-discharging tubes 1 and have a pattern in which the electrodes are configured to spread toward both sides of an electrode slit (electrode gap G) interposed between the electrodes.

If the insulating substrate 13 as the main body of the electrode substrate 11 is made of a polyimide resin-based insulating film, and the gas-discharging tubes 1 aligned are configured to have clearances therebetween, it is possible to make the surface-emitting ultraviolet light source device 10 flexible and curvable as a whole in a tube array direction. If the electrode substrate 11 has ventilation slits so as to partially expose bottom surfaces of the gas-discharging tubes 1 to the outside, the gas-discharging tubes 1 can restrain or regulate a rise in temperature, with the result that it is favorable to the gas-discharging tubes 1 to release heat and to be cooled.

The ventilation slits of the electrode substrate 11 facing the gas-discharging tubes 1, which are formed so that the bottom surface of each gas-discharging tube 1 is partially exposed to the outside, may be V-shaped or approximately U-shaped groove-shaped slits (non-penetrating type) or may be slits (ventilation holes) that penetrate the electrode substrate 11, in a direction perpendicular to the gas-discharging tube array direction. By providing the ventilation slits in the direction perpendicular to the tube array direction, heat generated from each gas-discharging tube 1 can be released to the outside almost evenly, and air can be blown almost evenly to each gas-discharging tube 1. This enhances the heat-releasing effect and/or the cooling effect of the surface-emitting ultraviolet light source device 10 and efficiently suppresses or controls an increase in temperature. As a result, a decrease in illuminance of the surface-emitting ultraviolet light source device 10 can be suppressed, and UV dose can be stabilized. When the heat-releasing mechanism or the cooling device 16 is provided in the configuration shown in FIG. 1A and FIG. 1B in which the electrode pair 12 is formed on the lower surface of the insulating substrate 13, the heat-releasing mechanism or the cooling device may be placed on the lower surface side of the insulating substrate 13, i.e., on the bottommost surface side of the electrode substrate 11. In any case, the heat-releasing mechanism or the cooling device is not limited to this configuration. When the heat-releasing mechanism or the cooling device 16 (the heat sink 17 and/or the Peltier element 18, etc.) is used, the electrode substrate 11 or the surface-emitting ultraviolet light source device 10 will be hereinafter referred to as including the heat-releasing mechanism or the cooling device 16 (the heat sink 17 and/or the Peltier element 18, etc.).

### Drive Principle

FIG. 1C shows a schematic view to describe a drive principle of the surface-emitting ultraviolet light source device 10. An inverter circuit 19 applies an alternating drive voltage to the electrode 12X and the electrode 12Y, which constitute the electrode pair 12, at a peak-to-peak voltage (P-P voltage) of 1,000 to 2,000 V at a frequency of 30 to 40 kHz. During an increasing process of the alternating drive voltage to be applied by the inverter circuit 19, an initial discharge is generated at a discharge gap inside the gas-discharging tubes 1, which is comparable to the electrode gap G between the electrode 12X and the electrode 12Y. Following the initial discharge, an electric discharge expands in a longitudinal direction of the gas-discharging tubes 1 as the alternating drive voltage increases.

By applying the alternating drive voltage, such an electric discharge occurs repeatedly while alternating polarity of a storage charge in regions inside the gas-discharging tubes 1 where correspond to the electrodes 12X, 12Y, respectively. In a case where a mixed gas of neon (Ne) and xenon (Xe) is enclosed in the gas-discharging tubes 1, an electric discharge is generated while vacuum ultraviolet (VUV) rays of 143 pnm and 172 pnm are emitted at a low discharge voltage compared to other gases. This VUV excitation causes the phosphor layer 3 to generate ultraviolet light and emit the ultraviolet light having a central wavelength of 203 inm through the glass tubes. This drive principle and the specific driving circuit are described in detail in the above-mentioned PTL 2.

### Second Embodiment

Hereinafter, Second Embodiment of the present invention will be described in detail with reference to the drawings.

As described above, the ultraviolet light irradiation device of the present invention is a device that does not basically require an optical filter; however, in order to further increase safety, an optical filter may be provided to the ultraviolet light irradiation device; and further, a timer may be provided so as to control driving time of the surface-emitting ultraviolet light source device.

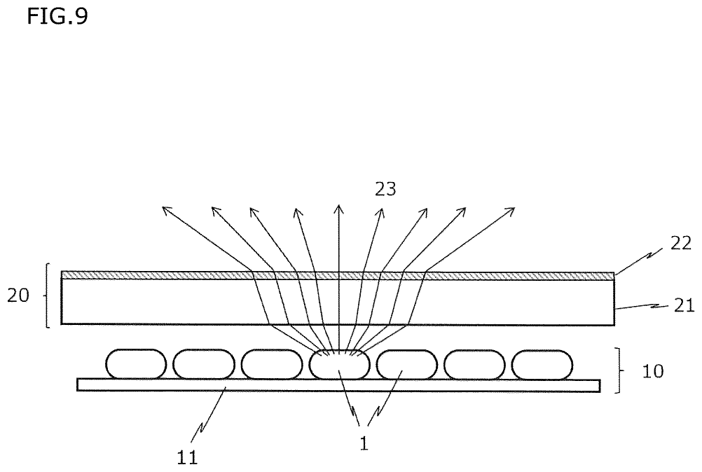

FIG. 9 shows a cross-section view of a structure of an ultraviolet light irradiation device in accordance with Second Embodiment. More specifically, FIG. 9 shows a cross-section view of a structure of an ultraviolet light irradiation device in which an optical filter 20 is assembled with the structure in accordance with First Embodiment shown in FIG. 1A to FIG. 1G and the surface-emitting ultraviolet light source device 10 of the present invention having the emission spectrum shown as 203B in FIG. 3.

The optical filter 20 assembled with the above-described structures may be used that is similar to the one proposed in PLT 5; however, it is desirable that the optical filter should have filter characteristics such that shorter wavelengths pass through the optical filter. Namely, the optical filter 20 is formed of a dielectric multilayer filter 22 with a thickness of the order of 1 to 2 μm, including an oxide film (for example, an oxide film containing hafnium oxide (molecular formula: HfO₂)/silicon dioxide (SiO₂)) formed on the ultraviolet light transmitting substrate—desirably the synthetic quartz substrate 21 with a thickness of the order of 1 mm. This optical filter 20 is characterized by transmitting mainly deep ultraviolet light with 190 to 230 inm and cutting off ultraviolet light with 240 inm or higher, which has a high potential to harm human tissue, i.e., effectively blocking the transmission of the above-mentioned ultraviolet light.

The deep ultraviolet light from the surface-emitting ultraviolet light source device 10 (which may be referred to as a gas-discharging tube array 10) to be used as a light source in the present invention has a wide radiation angle. On the other hand, the dielectric multilayer filter 22 to be assembled with the surface-emitting ultraviolet light source device 10 has incident angle dependence. In the configuration where radiant light from the surface-emitting ultraviolet light source device 10 is directly incident on the dielectric multilayer filter 22, a peripheral portion of ultraviolet light emitted at a wide angle results in insufficient transmission through the optical filter 20. This point is disclosed in PTL 5.

To maximize effectiveness of the optical filter 20 assembled with the surface-emitting ultraviolet light source device 10, the following can be done. In the present invention, in order to ensure that deep ultraviolet radiant light from the surface-emitting ultraviolet light source device 10 passes through the dielectric multilayer filter 22 as efficiently as possible, the dielectric multilayer filter 22 and the quartz substrate 21 are arranged in such a way that the quartz substrate 21 is positioned on the incident surface side of the optical filter 20. In such an arranged configuration, the quartz substrate 21 in front of the dielectric multilayer filter 22 functions as an optical element that converts the incident angle light rays emitted to the optical filter 20 at a wide radiation angle among the deep ultraviolet rays emitted from each gas-discharging tube 1 at a wide angle.

FIG. 9 shows, by using arrow lines 23, how radiant light rays are refracted from a light emitting surface of a gas-discharging tube 1 to the dielectric multilayer filter 22. The radiant light rays from the light emitting surfaces of the gas-discharging tubes 1, which constitute the surface-emitting ultraviolet light source device 10, enter the dielectric multilayer filter 22 at modified radiation angles in directions where incident angles are narrowed due to a difference between a refractive index (air n≈1) of a space, which is between the light source device and the quartz substrate 21, and a refractive index (n=1.5) of the quartz substrate 21. As a result, attenuation of radiant intensity of the deep ultraviolet light having a central wavelength of 203 inm emitted through the optical filter 20 can be suppressed.

To reduce loss of the incident angles of the deep ultraviolet radiant light 23 emitted at a wide angle from the surface-emitting ultraviolet light source device 10 toward the dielectric multilayer filter 22, the quartz substrate 21 constituting the optical filter 20 may further have ribs in a stripe pattern or a lattice pattern that function as a diffraction grating on the incident surface side. The stripe ribs corresponding to array pitches of the gas-discharging tubes 1 are capable of narrowing the incident angle of the deep ultraviolet light emitted from each gas-discharging tube 1 of the surface-emitting ultraviolet light source device 10 at a wide angle toward the dielectric multilayer filter 22.

Each of the glass tubules of the plurality of gas-discharging tubes 1 may further have a reflective layer that is formed on an outer surface on the back surface side facing the electrode substrate 11 or on the lateral surface side facing the adjacent glass tubule and reflects ultraviolet light from the inside of each glass tubule. By providing a metal film made of aluminum or the like placed on the adhesive layer 14 between the gas-discharging tubes 1 of the surface-emitting ultraviolet light source device 10, light emitted at a wide angle may be reflected forward to reduce an incident angle to the filter, thereby allowing the light to be emitted effectively forward. In this way, ultraviolet light can be emitted effectively forward even when a filter is not used.

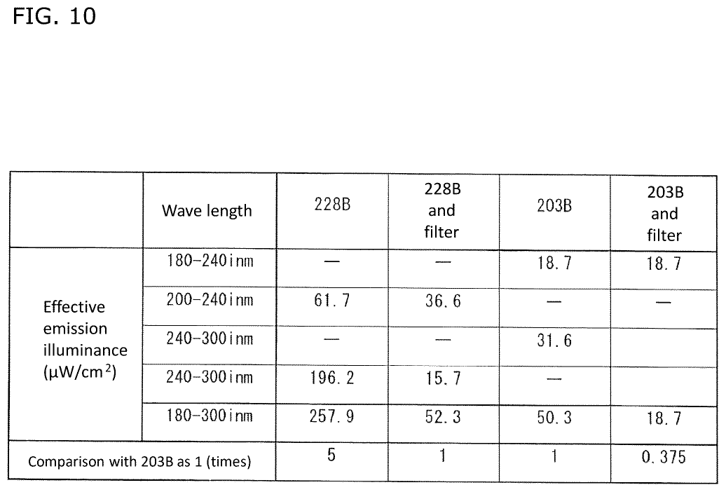

FIG. 10 is a comparison Table showing effectual irradiation illuminance in different categories, and the Table of FIG. 10 shows a relative comparison of different light sources and effective irradiation illuminance (indexes of degree of inhibition; unit: mJ/cm²). The different light sources include four (4) types: 228B; a combination of 228B and an optical filter; 203B; and a combination of 203B and an optical filter. FIG. 10 shows effective irradiation illuminance of the four (4) types of light sources: ultraviolet wavelengths of 180 to 240 inm (vacuum ultraviolet region); ultraviolet wavelengths of 200 to 240 inm (region considered less damage in a UVC region); ultraviolet of 240 to 300 inm; and ultraviolet of 180 to 300 inm. The optical filter used is the above-mentioned optical filter 20 shown in FIG. 9, which effectively cuts off (i.e., substantially blocks transmission of) ultraviolet light having wavelengths of 240 inm or more. The lower the effective radiation illuminance values, the higher the safety. The effective irradiation illuminance values in FIG. 10 are integrated values obtained by summing the emission spectrum in FIG. 3 and the action function in FIG. 4 for each wavelength. The action functions in FIG. 4 are unitless relative values. The spectra in FIG. 3 are shown as unitless relative values for comparison of waveforms; however, in the spectra where the vertical axis is expressed as illuminance, a unit is irradiation power per unit area, such as mW/cm² or μW/cm². In FIG. 10, calculated values with a unit of μW/cm² are shown.

As is clear from FIG. 10, the ultraviolet light irradiation device of the present invention using the above-described 203B light source alone is lower in effectual irradiation illuminance and safer at wavelengths of 180 to 300 inm even without an optical filter than an ultraviolet light irradiation device using the conventional 228B light source alone and an ultraviolet light irradiation device using both the 228B light source and an optical filter. In other words, the total effective irradiation illuminance at wavelengths from 180 to 300 inm is 50.3 μW/cm² when the 203B light source is used alone and 257.9 μW/cm² when the 228B light source is used alone. Therefore, it can be seen that the use of the 203B light source alone is safer. It is also clear from the comparison Table in FIG. 10 that the safety of the 203B light source can be further improved by using the optical filter 20 shown in FIG. 9. In this way, the 203B light source of the present invention can provide an ultraviolet light irradiation device with high safety even without using an optical filter. In particular cases where a high level of safety is required or where people with limited knowledge or experience in the handling of ultraviolet light use an ultraviolet light irradiation device or where an ultraviolet light irradiation device is used in environments where a higher safety of ultraviolet light irradiation is required, an extremely safe ultraviolet light irradiation device can be provided by using the optical filter 20 shown in FIG. 9. As shown in FIG. 10, the combination of the 203B light source and the optical filter is the safest among others, since the illuminance of UV light is 0 (zero) at wavelengths of 240 inm or higher.

The effective emission illuminance E_{eff} in FIG. 10 is a safety indicator set out in the guidelines published by the Global Lighting Association (GLA). The smaller the E_{eff}, the safer the light source is. The emission spectrum φ(λ) is calculated using an illuminance standard, and the effective emission illuminance E_{eff} is calculated by multiplying the emission spectrum φ(λ) by the action function S(λ) shown in FIG. 4. The effective emission illuminance E(λ) of wavelength λ can be obtained by the following equation:

$$E ( λ ) = φ ( λ ) \times S ( λ )$$

wherein
E(λ): effective emission illuminance
φ(λ): emission spectrum
S(λ): action function
Therefore, the effective emission illuminance of the target spectrum is obtained by integration of all spectral wavelengths at each wavelength.

In other words, the total effective emission illuminance can be obtained by the following equation:

$${E}_{eff} = \int φ ( λ ) \times S ( λ ) d λ$$

wherein
E_{eff}: total effective emission illuminance
φ(λ): emission spectrum
S(λ)dλ: action function

In the above equations, λ indicates a wavelength (measured based on an illuminance standard, and a unit of the wavelength is inm).

### Third Embodiment

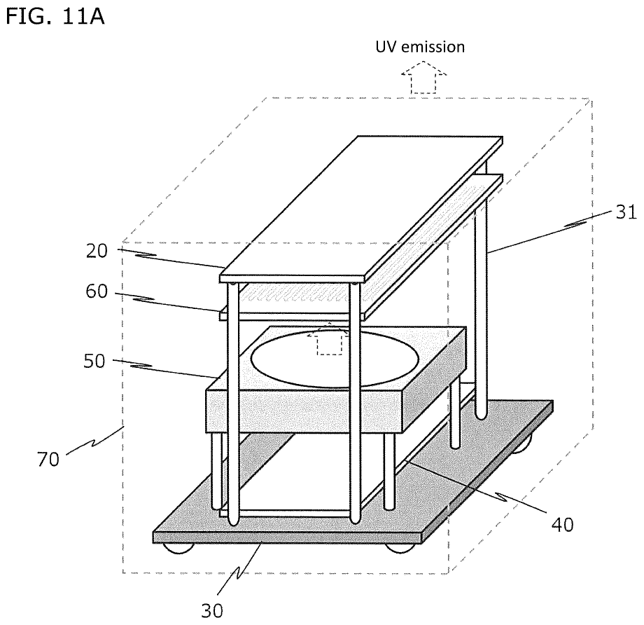

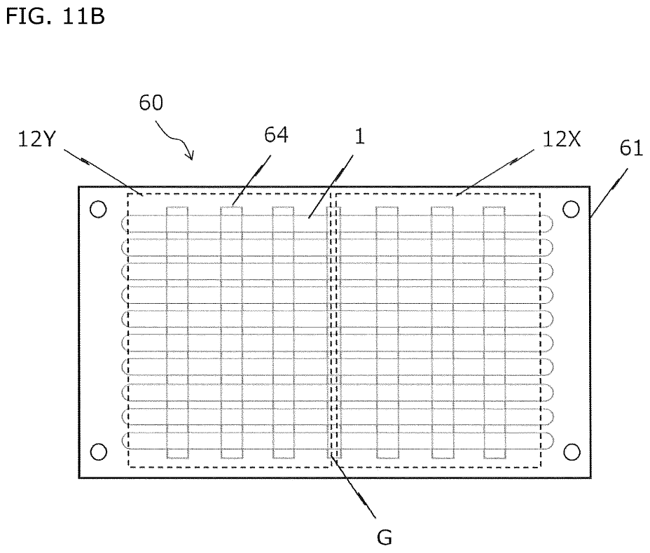

FIG. 11A shows a diagrammatic view of an assembled structure of an ultraviolet light irradiation device in accordance with Third Embodiment of the present invention. The ultraviolet light irradiation device shown in FIG. 11A comprises: a base substrate 30 with air inlet holes (not shown); a drive circuit board 40 placed on the base substrate 30; and an air-blowing fan 50 placed over the drive circuit board 40. This ultraviolet light irradiation device also comprises: a surface-emitting ultraviolet light source device 60 placed over the air-blowing fan 50; and an optical filter 20 placed, as needed, over the surface-emitting ultraviolet light source device 60. These (the surface-emitting ultraviolet light source device 60 and the optical filter 20) are assembled in this order from the bottom up and supported by four (4) columns 31. The surface-emitting ultraviolet light source device 60 is the same as any of the surface-emitting ultraviolet light source devices 10 shown in FIG. 1A to FIG. 1G and FIG. 9; and the optical filter 20 may be selectively provided to the ultraviolet light irradiation device depending on situations such as an environment in which the ultraviolet light irradiation device is used and a user of the device. The whole structure of the ultraviolet light irradiation device is placed in a housing 70 shown as a dotted diagram, with an ultraviolet irradiation window (aperture) at the top. In FIG. 11A, the ultraviolet light irradiation device is illustrated in such a way that a light emitting surface of the surface-emitting ultraviolet light source device 60 is configured, for convenience, to be nearly horizontal and also that UV radiation is directed upward; however, a direction of the UV radiation may be determined arbitrarily.

For the above-described surface-emitting ultraviolet light source device 60, an electrode substrate 61 is used having slits as shown in FIG. 11B. More specifically, like the electrode substrate 11, which has been described above with reference to FIG. 1B, the electrode substrate 61 shown in FIG. 11B has a plurality of through-slits 64 that are arranged almost in parallel with a clearance G functioning as a discharge gap and that penetrate through the electrodes 12X, 12Y as well as the electrode substrate 61. Such an electrode substrate 61 with the slits is configured in such a way that bottom surfaces (rear surfaces) of arrayed gas-discharging tubes 1 are partially exposed downward to the outside and that ventilation holes are formed at intersections of the following: clearances formed between the adjacent gas-discharging tubes; and the through-slits 64. This configuration allows cooling air from the air-blowing fan 50 to flow through the ventilation holes and thus is capable of effectively cooling the surface-emitting ultraviolet light source device 60. The ventilation holes formed in the electrode substrate 61 may be in the form of slits as described above or may be in the form of dispersed eyelets that are configured to partially expose the rear surfaces of the gas-discharging tubes.

### Fourth Embodiment

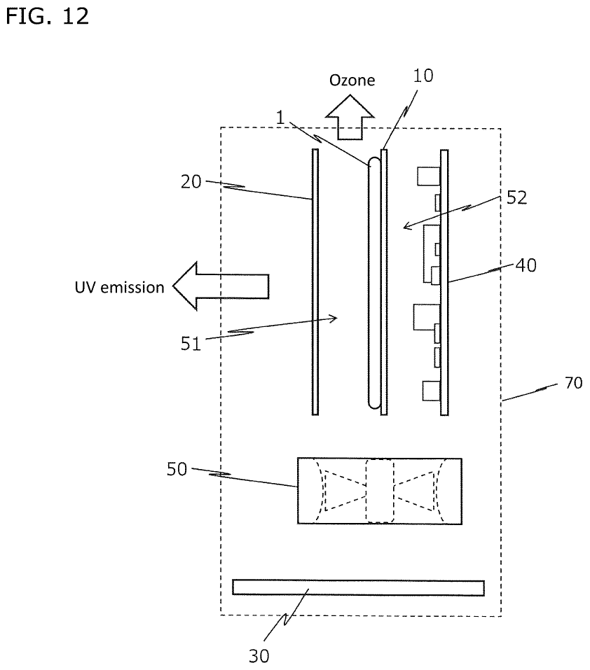

FIG. 12 shows a diagrammatic view of an assembled structure of an ultraviolet light irradiation device in accordance with Fourth Embodiment of the present invention, in which ozone is generated. The ultraviolet light irradiation device shown in FIG. 12 includes: a base substrate 30 having ventilation holes (which are omitted in the drawing); a gas-discharging tube array-type surface-emitting ultraviolet light source device 10 placed above the base substrate 30; an optical filter 20 placed on the front surface side (the light emitting surface side) of the surface-emitting ultraviolet light source device 10; a drive circuit board 40 placed on the rear surface side of the surface-emitting ultraviolet light source device 10; and a housing 70 (shown as a dotted diagram) housing these structures (the base substrate 30, the surface-emitting ultraviolet light source device 10, the optical filter 20, and the drive circuit board 40). The housing 70 has a window, through which ultraviolet light passes, on a wall facing the optical filter 20. The optical filter 20 may be placed optionally and may be omitted. There is a space, which functions as an ozone generation space 51, on the front surface side (the UV emission side) of the surface-emitting ultraviolet light source device 10 (the space placed between the light source device 10 and the optical filter 20 if the optical filter 20 is placed). There is a space, which functions as a heat-releasing passage 52, between the surface-emitting ultraviolet light source device 10 and the drive circuit board 40. In the housing 70, there is an air-blowing fan 50 on the opposite side of one end of the ozone generation space 51 and of the heat-releasing passage 52. The housing 70 has an outlet on a wall on the opposite side of the other end of the ozone generation space 51 and of the heat-releasing passage 52. When the air-blowing fan 50 is driven, ozone generated in the ozone generation space 51 is discharged to the outside from the outlet, and heat generated in the gas-discharging tube array 10 and the drive circuit board 40 is released to the outside from the outlet through the heat-releasing passage 52. Although the air-blowing fan 50 shown in FIG. 12 is disposed on the side (one end) of the surface-emitting ultraviolet light source device 10, the air-blowing fan may be disposed on the rear surface side of the light source device 10 and is not limited to the illustrated example of FIG. 12. The surface-emitting ultraviolet light source device 10 in accordance with Fourth Embodiment may be the same as the surface-emitting ultraviolet light source device 10 as in FIG. 1A to FIG. 1G or FIG. 9; and the optical filter 20 may be selectively provided to the ultraviolet light irradiation device depending on situations such as an environment in which the ultraviolet light irradiation device is used and a user of the device.

In Fourth Embodiment, as described above mainly with reference to FIG. 1A showing the gas-discharging tube 1, which is to constitute the surface-emitting ultraviolet light source device 10, the glass tubule 2 is made of highly UV-transmittable quartz glass or thin borosilicate-based glass. The short wavelength side of the vacuum ultraviolet radiation having a peak wavelength of 172 pnm generated by the electric discharge by xenon (Xe) gas in the tube 1 is absorbed by the glass of the glass tubule 2. The space inside each tube 1 is radiated with the vacuum ultraviolet radiation with a peak wavelength in the vicinity of 180 pnm. Therefore, in the space in the tube 1, the phosphor layer 3 simultaneously emits a deep ultraviolet emission spectrum (DUV) having a wavelength width of at least 180 to 230 pnm with a peak wavelength (first peak) around 210 pnm (203 inm) (210±10 pnm) and a vacuum ultraviolet (VUV) having a peak wavelength (second peak) around 180 pnm (180±10 pnm). FIG. 13 shows a graph showing emission spectrum characteristics of the 203B light source of the present invention. In FIG. 13, the horizontal axis indicates wavelength (pnm), and the vertical axis indicates the number of photons. FIG. 13 shows emission spectra of VUV (light A) and DUV (light B) from the gas-discharging tube array-type surface-emitting ultraviolet light source device of the 203B light source shown in FIG. 12. The phosphor layer 3 of the present Embodiment has the following features of the emission spectra (see FIG. 13): The wavelength of the first peak where illuminance becomes maximum with respect to wavelength is in a range of 203±10 inm (preferably 200 to 208 inm) on an illumination basis; the half value width of the first peak is in a wavelength range of 50 nm (see FIG. 3); the second peak is lower in illuminance than the first peak; the wavelength of the second peak is in a range of 180±10 pnm based on a photon quantity standard; and the spectral width at a 70% value with respect to a peak value of the second peak is in a wavelength range of 20 nm. Alternatively, the phosphor layer 3 of the present Embodiment has the following features of the emission spectra (see FIG. 13): The wavelength of the first peak where illuminance becomes maximum with respect to wavelength is in a range of 203±10 inm (preferably 200 to 208 inm) on an illumination basis; the half value width of the first peak is in a wavelength range of 50 nm (see FIG. 3); the second peak is lower in illuminance than the first peak; the wavelength of the second peak is in a range of 173±5 pnm based on a photon quantity standard; and the half value width of the second peak is in a wavelength range of 20 nm. Although not clearly illustrated in FIG. 13, the emission on the short wavelength side is absorbed due to wavelength transmission characteristics of glass and wavelength transmission characteristics of air; however, the peak at 180 pnm shifts to 173 pnm as the wavelength transmission characteristics are made more uniform (i.e., a transmission rate does not decrease even on the short wavelength side). For example, if the transmittance of the glass greatly decreases at 200 pnm or lower on the short wavelength side or if there is optical absorption on the short wavelength side due to oxygen in the air, the second peak at 180 pnm does not appear in the same way as light B as in FIG. 13. However, if a decrease in transmittance on the short wavelength side gradually eases, the second peak at 180 pnm begins to appear in the same way as light A. If transmittance on the short wavelength side is further increased, the second peak at 180 pnm shifts toward 173 pnm. If there is no effect from glass transmittance or light absorption by oxygen at all, this will in principle coincide with a molecular line emission peak of Xe gas.

FIG. 13 shows that vacuum ultraviolet light is absorbed by an air layer. That is, in FIG. 13, light A and light B indicate the spectra of the light emitting elements of the 203B measured in a nitrogen atmosphere and in an air atmosphere, respectively. In the nitrogen atmosphere, there is no absorption of vacuum ultraviolet light, and thus the light reaches the 170 pnm wavelength region to a photodetector about 5 mm away and is detected (light A). In the air atmosphere, light in the wavelength region of 190 pnm or less is not detected because the light is absorbed by oxygen between the air atmosphere and the photodetector and ozone is generated. In other words, the light in the wavelength region of 190 pnm or less is cut off (light B). The distance between a surface of the 203B shown in FIG. 12 and a measurement head (not shown) of the photodetector was set to 5 mm. In the 203B, the voltage applied to the inverter was 12 V, and the electric current was 1.4 A. Since there is no absorption of vacuum ultraviolet light in the nitrogen atmosphere, light up to a wavelength region of 170 pnm can reach the photodetector located about 5 mm away, and the spectrum of light A can be obtained. In the atmosphere, the light of 190 pnm or less is absorbed by oxygen by the time the light reaches the photodetector, and ozone is generated—i.e., the light of 190 pnm or less is cut off and is not detected—and thus the spectrum of light B is detected. Incidentally, hump-shaped peaks of 190 pnm or less are caused by reaction of the ozone and the light and absorption of the light. The difference between light A and light B contributes to generation of ozone.

The drive circuit board 40 is equipped with: a drive circuit including the inverter circuit 19 (see FIG. 1C) that is to supply an alternating drive voltage to the surface-emitting ultraviolet light source device 10; a timer for controlling exposure time (irradiation time); and a control circuit for controlling the whole ultraviolet light irradiation device. The surface-emitting ultraviolet light source device 10 driven by the drive circuit transmits deep ultraviolet light of 240 inm or less with a peak wavelength of about 203 inm through the optical filter 20 and irradiates air and/or a target object in an irradiation space with the ultraviolet light to sterilize or kill viruses/bacteria.

Of vacuum ultraviolet rays generated in the gas-discharging tube 1, vacuum ultraviolet rays with a wavelength of about 180 pnm that are reflected from the phosphor layer 3 having an ultraviolet reflection function are emitted from the light emitting surface. Of the vacuum ultraviolet rays, light with 190 pnm or less generates ozone by breaking down air in the ozone generation space 51 between the light emitting surface of the gas-discharging tube 1 constituting the surface-emitting ultraviolet light source device 10 and the optical filter 20. The principle of ozone generation by ultraviolet light has been known but will be briefly described here: Vacuum ultraviolet light (VUV: light A) generated in the gas-discharging tube 1 is emitted into air in the ozone generation space 51, which is outside the tube 1; and the vacuum ultraviolet light (VUV: light A) chemically reacts with oxygen in the air, generating ozone. More specifically, when oxygen molecules absorb photons, which are ultraviolet particles, the oxygen molecules dissociate into oxygen atoms; and these oxygen atoms bond with the oxygen molecules, forming ozone.

The ozone generated in the housing 70 is exhausted with air blown by the air-blowing fan 50 from the outlet to the outside, and exhibits a bactericidal action on the ambient air. Naturally, ozone itself has strong oxidation and toxicity and thus is greatly effective for elimination of viruses/bacteria and disinfection and deodorization in an unmanned enclosed space regardless of a concentration of the ozone; however, ozone must be limited from excess release into a manned environmental space. Ozone in low concentration, however, is harmless to the human body and is extremely useful for space sterilization (spatial disinfection). Therefore, ozone to be released into a manned space needs to be limited in concentration so as not to exceed a reference value of 0.1 ppm, which regulates ozone concentration. For example, a surface-emitting ultraviolet light source device 10 whose light emitting area is 8×3 cm in which twelve (12) gas-discharging tubes having a length of 8 cm are arranged is capable of generating 20 mg of ozone per hour. Such an amount of ozone generated from the surface-emitting ultraviolet light source device is capable of not only intermittently applying an alternating drive voltage and changing a duty ratio of the alternating drive voltage so as to appropriately control the amount of ozone but also controlling the drive by turning the drive on and off with feedback from a monitored value of the ozone concentration in the environmental space.

### Fifth Embodiment

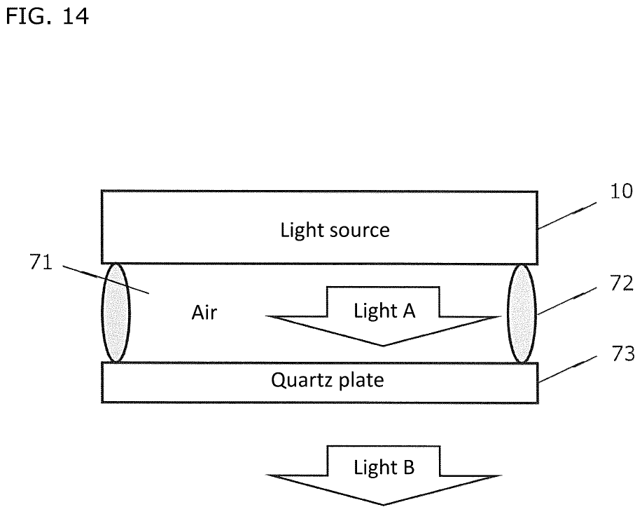

FIG. 14 shows a diagrammatic view of an assembled structure of an ultraviolet light irradiation device in accordance with Fifth Embodiment of the present invention. Ozone may be generated effectively in order to make active use of ozone as in Fourth Embodiment; however, if ozone is not to be used, its generation needs to be suppressed, given that ozone is harmful. The light source 10 shown in FIG. 14 is the same as the ones in the surface-emitting ultraviolet light source devices 10 used in the above-described Embodiments (see FIG. 1B and FIG. 9). In the UV irradiation device shown in FIG. 14, the optical filter 20 or a quartz plate 73 is placed on the front surface side of the light source 11—that is, the UV radiation side of the light source 11—with a void of about 5 mm. Along with that, a sealing material 72 is placed or coated around a perimeter between the light source 10 and the quartz plate 73, thereby forming an enclosed space 71 between the light source 10 and the quartz plate 73. The quartz plate 73 may or may not have a filtering function.

The effects of Fifth Embodiment will be described with reference to FIG. 13. As shown in FIG. 13, light A is an emission spectrum measured when an ultraviolet light source is placed in a nitrogen (N₂) atmosphere. The emission spectrum of light A has two peak wavelengths in the vicinity of 180 pnm and in the vicinity of 210 pnm, which are vacuum ultraviolet. Light B is an emission spectrum measured when the same light source is placed in an air atmosphere. The emission spectrum of light B rapidly attenuates at shorter wavelengths from around 190 pnm. This is because photons are absorbed by oxygen molecules and ozone is generated. In the ultraviolet light irradiation device according to Fifth Embodiment shown in FIG. 14, the enclosed space 71 filled with air is encapsulated, and ozone generated in the enclosed space 71 is also encapsulated by sealing a periphery between the light source 10 and the optical filter 20 (see FIG. 9) or between the light source and the quartz plate 73. At the same time, light (vacuum ultraviolet light) of 190 pnm or less is absorbed by an ozone layer in the enclosed space 71. Since the generated ozone does not leak out of the enclosed space 71, and the light of 190 pnm or less also does not leak out of the quartz plate 73, ozone is not generated outside the quartz plate 73. When ozone is not actively used, the irradiation device according to Fifth Embodiment can suppress or prevent harmful ozone from being released to the outside. In the case of this Embodiment shown in FIG. 14, it is necessary to form the enclosed space 71, and thus heat-releasing slits (through-holes) cannot be formed in the substrate of the light source 10. Slits, however, can be formed in the substrate of the light source 10 by doing the following: Preparing two UV-transmitting substrates, sealing a periphery of the two substrates after a space between the substrates is filled with air to form a panel, and placing this panel on the front surface side (the light emitting surface side) of the light source 10. This makes it possible to obtain the same effects (suppression of external emission of ozone) as in the case above.

### Conclusions Regarding Measures to Prevent Temperature Rise of 203B Light Source

FIG. 15A is a Table showing an increase in surface temperature of the 203B under various conditions. In the Table of FIG. 15A, exposure time (in seconds) is shown at the left end, and temperature (° C.) changes over time under conditions (1) through (7) are also shown. Conditions (1) to (7) in FIG. 15A are as follows:
(1) Substrate with slits placed on desk top. No cooling fan used.
(2) Substrate with slits placed 1.5 cm above desk. Air blown to the substrate from the side.
(3) Substrate with slits. Air blown to the substrate from the back.
(4) Substrate with slits. Ceramic heatsink placed on the back of the substrate.
(5) Substrate with slits. Aluminum heatsink placed on the back of the substrate.
(6) Substrate without slits. Aluminum heatsink placed on the back of the substrate.
(7) Substrate without slits. Air blown to the substrate from the back.
   FIG. 15B is a graph based on numerical values shown in the Table of FIG. 15A; and the horizontal axis indicates exposure time (seconds), and the vertical axis indicates temperature (° C.). The data in FIG. 15A and FIG. 15B were obtained by using the surface-emitting ultraviolet light source device 10 shown in FIG. 1B and employing various heat-releasing methods or cooling methods to find effects of each method and effects of suppressing temperature rise. As shown in conditions (1) to (7) in FIG. 15A, the heat-releasing methods or the cooling methods include the following features: having slits in the electrode substrate; blowing air by using a fan; and having a ceramic or aluminum heat sink, and these features may be included either alone or in combination. Note that conditions (1) to (7) do not use the Peltier device shown in FIG. 1F and FIG. 1G.

As is clear from the results shown in FIG. 15A and FIG. 15B, it was ascertained that under conditions (2) to (6), the temperature rise can be reduced (limited) to about 10 to about 15° C. even after substantially about 4 minutes of continuous operation of the LAFi-driven 203B light source shown in FIG. 1B. In view of temperature and illuminance characteristics shown in FIG. 8, a decrease in illuminance due to a 10° C. temperature rise can be suppressed (limited) to about 10% by adopting conditions (2) to (6), and it was found that this is practically not a practical problem. Such a combination of the heat-releasing and the cooling makes it possible to secure the exposure time even if the illuminance is reduced slightly in order to obtain a dose (which is the product of UV intensity (mW/cm²) and exposure time (seconds)) required as the UV dose of the surface-emitting ultraviolet light source device 10; therefore, it is not necessary to, for example, control the exposure time while managing the temperature.

### CONCLUSIONS

By virtue of the present invention, a safe ultraviolet light irradiation device can be obtained that has wide elimination/disinfection effects on viruses/bacteria (microorganisms) with the use of ultraviolet light that is safe to the human body without using an expensive and high performance optical filter. Also, the present invention may provide an ultraviolet disinfection device provided with the ultraviolet light irradiation device and may also provide an ultraviolet disinfection lighting device provided with the ultraviolet light irradiation device and a lighting device. This invention has another aspect that includes any combinations of the above-described aspects. In addition to the above-described Embodiments, this invention may have a variety of modified examples. It should not be interpreted that these modified examples are excluded from the scope of this invention. The scope of this invention intends to include the meanings equivalent to the scope of the claims and also include all alterations (and modifications) within the scope of the claims.

### REFERENCE SIGNS LIST

1: gas-discharging tube
2: glass tubule
3: phosphor layer
4: discharge gas
10, 60: surface-emitting ultraviolet light source device, light source device, gas-discharging tube array, light source
11, 61: electrode substrate
12: a pair of electrodes
12X, 12Y: electrode
13: insulating substrate
14: adhesive layer
15: electrode-covering layer (insulating layer)
16: heat-releasing mechanism, cooling device
17: heat sink
18: Peltier element
19: inverter circuit
20: optical filter
21: quartz substrate, synthetic quartz substrate
22: dielectric multilayer filter
23: radiant light, arrow line
30: base substrate
31: column
40: drive circuit board
50: air-blowing fan
51: ozone generation space
52: heat-releasing passage
64: through-slit
70: housing
71: enclosed space
72: sealing material
73: quartz plate
181: heat-releasing plate
182: heat-absorbing plate
183: n-type semiconductor
184: p-type semiconductor
185: power supply line
G: electrode gap

What is claimed is:

1.-23. (canceled)

24. An ultraviolet light emitting element provided with:
an electrode substrate having a pair of electrodes;
at least one cylindrical or flattened cylindrical glass tube disposed on the electrode substrate so as to face both of the electrodes, the glass tube having both end parts sealed;
a xenon gas-containing discharge gas enclosed inside the glass tube and generating an electric discharge due to a voltage applied to the electrodes; and
a phosphor layer formed on an inner surface of the glass tube and emitting light when excited by the electric discharge,
wherein the glass tube is made of borosilicate glass or quartz glass, and
the phosphor layer contains ScPO4 as a fluorescent material and has an emission spectrum having a peak half value width within a wavelength range of 50 nm or less with a peak at a wavelength of 203±10 inm on an illuminance basis.

25. The ultraviolet light emitting element according to claim 24, wherein
the glass tube is made of the quartz glass,
the light generated from the phosphor layer and emitted to the outside of the glass tube is in a range of 203±10 inm on an illuminance basis, which has a peak wavelength where illuminance becomes maximum; and the light has an emission spectrum having a peak half value width within a wavelength range of 50 nm.

26. The ultraviolet light emitting element according to claim 24, wherein
the glass tube made of the borosilicate glass transmits 80% or more of light generated inside the glass tube at a wavelength of 200 pnm on a photon basis,
the phosphor layer has an emission spectrum in which a wavelength of a first peak where illuminance becomes maximum with respect to wavelength is in a range of 203±10 inm on an illuminance basis; a half value width of the first peak is in a wavelength range of 50 nm; a second peak is lower in illuminance than the first peak; a wavelength of the second peak is in a range of 180±10 pnm on a photon quantity basis; and a spectral width at a 70% value with respect to a peak value of the second peak is in a wavelength range of 20 nm.

27. The ultraviolet light emitting element according to claim 25, wherein the peak wavelength is in a range of 200 to 208 inm on an illuminance basis.

28. The ultraviolet light emitting element according to claim 26, wherein the wavelength of the first peak is in a range of 200 to 208 inm on an illuminance basis.

29. The ultraviolet light emitting element according to claim 25, wherein luminescence intensity of the light emitted from the phosphor layer at a wavelength of 250 inm is 10% or less of luminescence intensity of the peak wavelength on an illuminance basis.

30. The ultraviolet light emitting element according to claim 26, wherein luminescence intensity of the light emitted from the phosphor layer at a wavelength of 250 inm is 10% or less of luminescence intensity of the first peak wavelength on an illuminance basis.

31. The ultraviolet light emitting element according to claim 25, wherein luminescence intensity of the light emitted from the phosphor layer in a wavelength region of 240 inm or more is 20% or less of luminescence intensity of the peak wavelength on an illuminance basis.

32. The ultraviolet light emitting element according to claim 26, wherein luminescence intensity of the light emitted from the phosphor layer in a wavelength region of 240 inm or more is 20% or less of luminescence intensity of the first peak on an illuminance basis.

33. An ultraviolet light irradiation device comprising:
the ultraviolet light emitting element according to claim 24 having a plurality of glass tubes, each glass tube identical to the glass tube according to claim 24; and
a heat-releasing mechanism or a cooling device that is placed on the rear surface side of the ultraviolet light emitting element and suppresses a temperature rise of each glass tube during light emission.

34. The ultraviolet light irradiation device according to claim 33, wherein each of the plurality of glass tubes further has a reflective layer that is formed on an outer surface on the back surface side facing the electrode substrate or on the lateral surface side facing the adjacent glass tube and that reflects ultraviolet light from the inside of each glass tube.

35. The ultraviolet light irradiation device according to claim 33, wherein the heat-releasing mechanism or the cooling device has slits that penetrate the electrode substrate.

36. The ultraviolet light irradiation device according to claim 33, wherein the heat-releasing mechanism or the cooling device is provided with a heat sink, which is made of ceramic or aluminum, installed on the back of the electrode substrate.

37. The ultraviolet light irradiation device according to claim 33, wherein the heat-releasing mechanism or the cooling device is provided with a Peltier element or a vapor chamber that is attached to the back of the electrode substrate.

38. An ultraviolet light emitting element provided with:
an electrode substrate having a pair of electrodes;
at least one cylindrical or flattened cylindrical glass tube disposed on the electrode substrate so as to face both of the electrodes, the glass tube having both end parts sealed;
a xenon gas-containing discharge gas enclosed inside the glass tube and generating an electric discharge due to a voltage applied to the electrodes; and
a phosphor layer formed on an inner surface of the glass tube and emitting light when excited by the electric discharge,
wherein the glass tube made of the borosilicate glass transmits 80% or more of light generated inside the glass tube at a wavelength of 200 pnm and transmits 45% or less of light at a wavelength of 180 pnm, on a photon quantity basis,
the light generated from the phosphor layer and emitted to the outside of the glass tube is in a range of 203±10 inm on an illuminance basis, which has a peak wavelength where illuminance becomes maximum; and the light has an emission spectrum having a peak half value width within a wavelength range of 50 nm.

39. An ultraviolet light emitting element provided with:
an electrode substrate having a pair of electrodes;
at least one cylindrical or flattened cylindrical glass tube disposed on the electrode substrate so as to face both of the electrodes, the glass tube having both end parts sealed;
a xenon gas-containing discharge gas enclosed inside the glass tube and generating an electric discharge due to a voltage applied to the electrodes; and
a phosphor layer formed on an inner surface of the glass tube and emitting light when excited by the electric discharge,
wherein the glass tube is made of the quartz glass,
the phosphor layer has an emission spectrum in which a wavelength of a first peak where illuminance becomes maximum with respect to wavelength is in a range of 203±10 inm on an illuminance basis; a half value width of the first peak is in a wavelength range of 50 nm; a second peak is lower in illuminance than the first peak; a wavelength of the second peak is in a range of 173±5 pnm on a photon quantity basis; and a half value width of the second peak is in a wavelength range of 20 nm.

40. The ultraviolet light emitting element according to claim 38, wherein the phosphor layer has an emission spectrum in which a wavelength of a first peak where illuminance becomes maximum with respect to wavelength is in a range of 203±10 inm on an illuminance basis; a half value width of the first peak is in a wavelength range of 50 nm; a second peak is lower in illuminance than the first peak; a wavelength of the second peak is in a range of 180±10 pnm on a photon quantity basis; and a spectral width at a 70% value with respect to a peak value of the second peak is in a wavelength range of 20 nm.

41. The ultraviolet light emitting element according to claim 39, wherein the light generated from the phosphor layer and emitted to the outside of the glass tube is in a range of 203±10 inm on an illuminance basis, which has a peak wavelength where illuminance becomes maximum; and the light has an emission spectrum having a peak half value width within a wavelength range of 50 nm.

42. The ultraviolet light emitting element according to claim 38, wherein the phosphor layer contains ScPO₄ as a fluorescent material.

43. An ultraviolet light irradiation device comprising:
the ultraviolet light emitting element according to claim 38 having a plurality of glass tubes, each glass tube identical to the glass tube according to claim 38; and
a heat-releasing mechanism or a cooling device that is placed on the rear surface side of the ultraviolet light emitting element and suppresses a temperature rise of each glass tube during light emission.

44. An ultraviolet light irradiation device comprising:
the ultraviolet light emitting element according to claim 39 having a plurality of glass tubes, each glass tube identical to the glass tube according to claim 39; and
a heat-releasing mechanism or a cooling device that is placed on the rear surface side of the ultraviolet light emitting element and suppresses a temperature rise of each glass tube during light emission.
